(19) United States
(12) Patent Application Publication
(10) Pub. No.: US 2022/0002438 A1
(43) Pub. Date: Jan. 6, 2022

(54) MUSK INHIBITION

(71) Applicant: ACADEMISCH ZIEKENHUIS LEIDEN (H.O.D.N. LUMC), Leiden (NL)

(72) Inventors: Silvère Maria VAN DER MAAREL, Leiden (NL); Johannes Justus Gerard Maria VERSCHUUREN, Leiden (NL); Martina Gerardina Maria HUIJBERS, Leiden (NL); Jakob Jan PLOMP, Leiden (NL)

(21) Appl. No.: 17/272,791

(22) PCT Filed: Sep. 5, 2019
(86) PCT No.: PCT/NL2019/050576

§ 371 Date: Mar. 2, 2021

(30) Foreign Application Priority Data
Sep. 10, 2018 (NL) 2021589
May 13, 2019 (NL) 2023119

(51) Int. Cl. C07K 16/40

(52) U.S. Cl. CPC C07K 16/40; C07K 2317/35; C07K 2317/75; C07K 2317/24; C07K 2317/76; C07K 2317/21

(57) ABSTRACT

Novel methods for treating condition, disorder and/or symptom which is alleviated by the inhibition of neuromuscular transmission in a subject are provided herein. The invention also provides binding agents for use in treating the same.

Novel methods for treating condition, disorder and/or symptom which is alleviated by the inhibition of MuSK-mediated neuromuscular transmission in a subject are provided herein. The invention also provides binding agents for use in treating the same.

### BACKGROUND

The neuromuscular junction (NMJ) is the location where the motor neuron makes contact with the muscle fibre. At this site, the motor neuron and muscle fibre together create a specialized structure that enables communication between the two cells in both directions. The motor neuron instructs the muscle to contract. Several signal transduction cascades are involved to regulate this communication process. At the centre of neuromuscular transmission is the release of acetylcholine (ACh) by the motor neuron ending. ACh diffuses through the synaptic cleft to the muscle fibre. There it binds ACh receptors (AChR) that are located at the muscle fibre membrane. This results in a muscle membrane action potential and ultimately muscle fibre contraction.

The release of sufficient ACh and the presence of densely clustered AChR are prerequisites for successful neuromuscular transmission and muscle contraction. The agrin—low density lipoprotein receptor-related protein 4 (Lrp4)—muscle-specific kinase (MuSK) signalling cascade is an important regulator of AChR clustering. This signalling cascade is important for establishing and maintaining neuromuscular synapses. MuSK transduces the extracellular signal internally to facilitate AChR clustering. ACh release and binding to the AChR mediates muscle contraction.

A number of different agents that affect neuromuscular transmission are known and are regularly used in a cosmetic and/or therapeutic setting. A popular example is botulinum neurotoxin, which is currently used for several therapeutic and non-therapeutic applications (e.g. for reducing wrinkles, or treating muscle spasms). However, due to the extreme toxicity and inherent immunogenicity of these toxins, their use is typically limited to administration in extremely low doses to avoid or minimise side effects such as unwanted paralysis and/or a host immune response (Naumann, M., Boo, L. M., Ackerman, A. H. & Gallagher, C. J. J Neural Transm (Vienna) 120, 275-290, (2013)).

Botulinum neurotoxin type A (BoNT/A) is the active substance in preparations that are currently used for the treatment of several conditions, disorders and/or symptoms which are alleviated by the inhibition of neuromuscular transmission. Botulinum toxin is a biological product derived from bacterium Clostridium botulinum type A. In most therapeutics BoNT/A is part of a complex with other proteins. Therefore, changes in the composition of these products cannot be excluded (see for example the content of botulinum neurotoxin in Botox®/Vistabel®, Dysport®/Azzalure®, and Xeomin®/Bocouture®. Frevert J. Drugs R D. 2010; 10(2):6 7-73).

Treatment with botulinum toxin is also known to have a number of side effects. Such side effects include transient fatigue, dysphagia, neck weakness, hoarseness and localized pain. In addition, many individuals that preliminarily respond to botulinum toxin therapy subsequently become non-responsive to the treatment. Accordingly, for many individuals the botulinum injections fail to provide satisfactory long-term treatment of the condition.

There is a need for better blockers of neuromuscular transmission for cosmetic and/or therapeutic applications.

### SUMMARY OF THE INVENTION

The inventors have isolated and characterized several MuSK monoclonal antibodies from autoimmune myasthenia gravis (MG) patients and examined their functional characteristics to further understand the pathomechanism of MuSK MG.

MG is the most common disorder of the neuromuscular synapse, affecting 10 to 20 per 100,000 people in the US. It is a debilitating autoimmune disease where autoantibodies against NMJ proteins impair neuromuscular transmission and cause fatigable muscle weakness. All skeletal muscles can be affected although, depending on MG subtype, specific subsets of muscles are more sensitive to the autoimmune attack. Approximately 80% of patients carry autoantibodies against muscle nicotinic AChR, resulting in AChR MG. In most patients the first symptom is extraocular muscle weakness, and when the disease progresses, also bulbar or generalized weakness of skeletal muscles occurs. The pathomechanism by which AChR autoantibodies cause MG is tightly related to the autoantibody isotype; immunoglobulin (IgG) 1 and IgG3. IgG1 and IgG3 are pro-inflammatory antibodies which can activate complement, bind Fc receptors on immune cells and crosslink and internalize the antigen. In addition, these AChR antibodies can crosslink AChRs and cause antigen modulation of the AChRs, block the binding site of ACh, or change the affinity for ACh by inducing structural changes of the AChR. These effector functions are all contributing to the disease in AChR MG by diminishing the availability of functional AChRs and result in functional impairment and disassembly of the NMJ.

Approximately 5% of MG patients have autoantibodies against MuSK, resulting in MuSK MG (Hoch et al., 2001). MuSK orchestrates AChR clustering and sub-synaptic gene expression and is therefore essential for NMJ formation and maintenance (Burden et al., 2018). Bulbar and respiratory muscles are particularly affected by MuSK autoantibodies which can lead to respiratory crisis in approximately 40% of these patients (Evoli et al., 2003). In contrast to AChR MG, the autoantibodies in MuSK MG are predominantly of the IgG4 isotype (McConville et al., 2004). Epitope mapping with polyclonal serum antibodies showed that disease severity correlates with IgG4 reactivity against the N-terminal Ig-like 1 domain of MuSK (Huijbers et al., 2016). Furthermore, passive transfer of purified human polyclonal IgG4, but not IgG1-3, from MuSK MG patients confirmed the pathogenic nature of these autoantibodies as they induced MG in immunocompromised mice (Klooster et al., 2012). In vitro studies showed that MuSK IgG4 autoantibodies purified from plasma block MuSK-Lrp4 interaction thereby inducing AChR declustering which culminates in impaired neuromuscular transmission and MG (Huijbers & Zhang 2013, Koneczny et al., 2013, Otsuka et al., 2015).

The inventors have generated recombinant MuSK antibodies from clonal MuSK-specific memory B cell cultures from MuSK MG patients to characterize them on a genetic and functional level. These included IgG1, IgG3 and IgG4 antibodies that used different heavy and light chain variable region genes that had undergone high levels of affinity maturation in their complementary-determining regions (CDRs), consistent with antigenic selection. Binding experiments confirmed their specificity for the Ig-like 1 domain of MuSK and their affinity for mouse NMJs.

It is known that IgG4 autoantibodies that bind to the Ig-like 1 domain of MuSK can cause the disease of MuSK MG. IgG4 is initially generated in vivo in bivalent, monospecific form (i.e. with two variable regions specific for the same target antigen). IgG4 then undergoes exchange of half-IgGs over time (a process called Fab-arm exchange in the literature and herein, see below), resulting in a pool of bivalent bi-specific IgG4 molecules in vivo (i.e. with two variable regions specific for different target antigens). Over time in vivo, IgG4 therefore becomes functionally monovalent for its original target antigen (i.e. although it still has two variable regions, it retains only one variable region that specifically binds to the original target; the Ig-like 1 domain of MuSK in this instance). This is a rapid process occurring within 24 hours for >99% of the IgG4 pool (provided that all requirements are met for Fab-arm exchange among which that the required residues are present in the IgG4 Fc tail).

The inventors have surprisingly found that MuSK antibodies only inhibit agrin-induced MuSK phosphorylation (and thus inhibit MuSK dimerization and activation) when they are functionally monovalent for the Ig-like 1 domain of MuSK (i.e. when they retain only one variable region that specifically binds to the Ig-like 1 domain of MuSK). The data provided herein explains, for the first time, that the IgG4 autoantibodies observed in MuSK MG patients only become inhibitory in nature when they become functionally monovalent (thus in vivo after they have undergone Fab-arm exchange). The ability of patient IgG4 MuSK antibodies to undergo Fab-arm exchange seems therefore crucial for the pathogenesis of disease as Fab-arm exchange renders endogenous MuSK IgG4 bi-specific and functionally monovalent for the MuSK Ig-like 1 domain. Only in this state does the IgG4 act as an inhibitor of MuSK activity.

The inventors have also surprisingly shown that agrin-independent MuSK phosphorylation is induced or increased when the MuSK antibodies are bivalent and monospecific for the Ig-like 1 domain of MuSK (i.e. when they have two variable regions that both specifically bind to the Ig-like 1 domain of MuSK). The inventors have therefore shown that the valency of an antibody for the Ig-like 1 domain of MuSK determines whether the antibody is a MuSK antagonist (bivalent bi-specific antibody, or monovalent monospecific antibody fragment) or an agonist (bivalent, monospecific antibody).

The invention is completely surprising, as the data presented herein shows that agonist bivalent monospecific MuSK Ig-1 like domain antibodies can be converted into antagonists for the same target antigen when forced to retain only one variable region that specifically binds to the Ig-like 1 domain of MuSK.

In one aspect, a binding agent comprising one or more binding regions, wherein only one binding region specifically binds to an Ig-like 1 domain of a MuSK protein, is provided for use in treating a condition, disorder and/or symptom which is alleviated by the inhibition of neuromuscular transmission in a subject.

Advantageously, the binding agent described herein does not induce (e.g. is not capable of inducing) MuSK dimerization and/or phosphorylation and/or activation.

Suitably, the binding agent is a binding protein.

Suitably, the region that specifically binds to an Ig-like 1 domain of the MuSK protein is a variable region.

Suitably, the MuSK protein is a human MuSK protein.

Suitably, the binding agent is monovalent.

Suitably, the binding agent is bivalent or trivalent. In this context, the bivalent or trivalent binding agent does not induce (e.g. is not capable of inducing) MuSK dimerization or activation.

Suitably, the binding agent is an antibody.

Suitably, the antibody is a monoclonal antibody.

Suitably, the antibody is a human antibody or a humanised antibody.

Suitably, the antibody is selected from a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody.

Suitably, the antibody is an IgG. The IgG may be selected from IgG1, IgG2 or IgG3. Alternatively, the IgG may be an IgG4 variant with a reduced ability for or an inability for Fab-arm exchange in vivo.

Suitably, the IgG4 variant comprises an IgG4 constant region comprising one or more amino acid substitutions that reduce the ability for or prevent Fab-arm exchange in vivo.

Suitably, the IgG4 variant comprises an IgG4 constant region comprising an amino acid substitution at amino acid position 228 and/or an amino acid substitution at amino acid position 409 and/or an amino acid substitution at amino acid position 405 of the heavy chain numbered according to the EU index.

Suitably, the subject is a human.

Suitably, the condition, disorder and/or symptom is selected from:

(i) an external appearance distorted due to excessive muscular activity; or

(ii) a condition, disorder or symptom resulting from excessive muscular activity, including dystonias, facial spasms, strabismus, cerebral palsy, stuttering, chronic tension headaches, spasms of the inferior constrictor of the pharynx, pain, migraine, involuntary spasms, muscle spasticity, strabismus, occupational cramps, anal fissures, bruxism, and any combination thereof.

Suitably, the only one binding region that specifically binds to an Ig-like 1 domain of a MuSK protein has a sequence selected from:

a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO:13;

b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;

c) a VH CDR1 comprising SEQ ID NO:26, a VH CDR2 comprising SEQ ID NO:27, and a VH CDR3 comprising SEQ ID NO:28, optionally wherein the binding region comprises a heavy chain variable domain comprising SEQ ID NO: 25;

d) a VH CDR1 comprising SEQ ID NO:30, a VH CDR2 comprising SEQ ID NO:31, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;

e) a VH CDR1 comprising SEQ ID NO:38, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;

f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;

g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or

h) combinations of any of the above.

A method of preventing, regulating or reducing skin wrinkling in a subject is also provided, the method comprising administering a binding agent comprising one or more binding regions, wherein only one binding region specifically binds to an Ig-like 1 domain of a MuSK protein, to the subject.

A method of treating or preventing a condition, disorder and/or symptom which is alleviated by the inhibition of neuromuscular transmission is also provided; the method comprising administering a binding agent comprising one or more binding regions, wherein only one binding region specifically binds to an Ig-like 1 domain of a MuSK protein, to a subject.

Suitably, the condition, disorder and/or symptom is selected from:

(i) an external appearance distorted due to excessive muscular activity; or

(ii) a condition, disorder or symptom resulting from excessive muscular activity, including dystonias, facial spasms, strabismus, cerebral palsy, stuttering, chronic tension headaches, spasms of the inferior constrictor of the pharynx, pain, migraine, involuntary spasms, muscle spasticity, strabismus, occupational cramps, anal fissures, bruxism, and any combination thereof.

Advantageously, the binding agent described herein does not induce (e.g. is not capable of inducing) MuSK dimerization and/or phosphorylation and/or activation.

Suitably, the binding agent is a binding protein.

Suitably, the region that specifically binds to an Ig-like 1 domain of the MuSK protein is a variable region.

Suitably, the MuSK protein is a human MuSK protein.

Suitably, the binding agent is monovalent.

Suitably, the binding agent is bivalent or trivalent. In this context, the bivalent or trivalent binding agent does not induce (e.g. is not capable of inducing) MuSK dimerization or activation.

Suitably, the binding agent is an antibody.

Suitably, the antibody is a monoclonal antibody.

Suitably, the antibody is a human antibody or a humanised antibody.

Suitably, the antibody is selected from a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody.

Suitably, the antibody is an IgG. The IgG may be selected from IgG1, IgG2 or IgG3. Alternatively, the IgG may be an IgG4 variant with reduced ability for or an inability for Fab-arm exchange in vivo.

Suitably, the IgG4 variant comprises an IgG4 constant region comprising one or more amino acid substitutions that reduce or prevent the ability for Fab-arm exchange in vivo.

Suitably, the IgG4 variant comprises an IgG4 constant region comprising an amino acid substitution at amino acid position 228 and/or an amino acid substitution at amino acid position 409 and/or an amino acid substitution at amino acid position 405 of the heavy chain numbered according to the EU index.

Suitably, the subject is a human.

Suitably, the only one binding region that specifically binds to an Ig-like 1 domain of a MuSK protein has a sequence selected from:

a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO:13;

b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;

c) a VH CDR1 comprising SEQ ID NO:26, a VH CDR2 comprising SEQ ID NO:27, and a VH CDR3 comprising SEQ ID NO:28, optionally wherein the binding region comprises a heavy chain variable domain comprising SEQ ID NO: 25;

d) a VH CDR1 comprising SEQ ID NO:30, a VH CDR2 comprising SEQ ID NO:31, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;

e) a VH CDR1 comprising SEQ ID NO:38, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;

f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;

g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or

h) combinations of any of the above.

In another aspect, an antibody is provided comprising one or more binding regions, wherein only one binding region specifically binds to an Ig-like 1 domain of a MuSK protein, wherein the only one binding region that specifically binds to the Ig-like 1 domain of a MuSK protein has a sequence selected from:

a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO:13;

b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;

c) a VH CDR1 comprising SEQ ID NO:26, a VH CDR2 comprising SEQ ID NO:27, and a VH CDR3 comprising SEQ ID NO:28, optionally wherein the binding region comprises a heavy chain variable domain comprising SEQ ID NO: 25;

d) a VH CDR1 comprising SEQ ID NO:30, a VH CDR2 comprising SEQ ID NO:31, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;

e) a VH CDR1 comprising SEQ ID NO:38, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;

f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;

g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or

h) combinations of any of the above.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

Embodiments of the invention are further described hereinafter with reference to the accompanying drawings, in which:

FIG. 1 shows that patient derived-recombinant MuSK antibodies bind mouse NMJs in whole mount levator auris longus muscle. Scale marker is 25 um.

FIG. 2 shows that patient-derived recombinant MuSK antibodies can activate or inhibit MuSK phosphorylation and AChR clustering depending on the antibody valency. Bivalent monospecific recombinant MuSK antibodies (Clone #11-3F6 and 13-3B5) activated MuSK phosphorylation in the presence and absence of agrin (A). Activation of MuSK phosphorylation was dose-dependent (B). Clone 13-3B5 was slightly more potent compared to 11-3F6. A biotin control antibody did not affect (agrin-dependent) MuSK phosphorylation. Monovalent Fab fragments generated from these recombinant MuSK monoclonals inhibited MuSK phosphorylation (C). Agrin-dependent AChR clustering was unaffected when exposed to a biotin control antibody or Fab fragments thereof (D). Bivalent monospecific recombinant IgG4 monoclonal MuSK antibodies significantly increased AChR clustering compared to Fab fragments derived from the same monoclonal, although agrin-dependent AChR clustering remained lower than the anti-biotin control (for IgG4: P=0.0004, for Fab fragments: P=0.0001, one-way ANOVA Dunnett corrected, 11-3F6 IgG4 vs Fab: P=0.0003, 13-3B5 IgG4 vs Fab: P=0.03, anti-biotin IgG4 vs Fab: P=0.3, unpaired t test). Fab fragments reduced AChR cluster to the level of purified patient IgG4 and the “no agrin” condition. Bivalent monospecific antibodies furthermore significantly increased AChR clustering independent from agrin (11-3F6: P=0.03, 13-3B5: P=0.2 one-way ANOVA Dunnett corrected). Data represent mean±SEM. Scale bar represents 50 μm.

FIG. 3 shows MuSK reactivity results for 8 B cell clones isolated from MuSK MG patients. MuSK was produced in yeast and E. Co/i. MuSK reactive B cell clones isolated from MuSK MG patients and healthy donors were single cell sorted and differentiated to plasma cells. Medium from these single cell cultures was tested in ELISA for MuSK (produced in E. Coli.) reactivity using subclass-(a)specific (anti-IgG1 or anti-IgG4 or anti-IgG total) secondary antibodies. In these experiments 8 MuSK reactive B cell clones were isolated, of which 7 yielded a B cell receptor (BCR) sequence. The level of reactivity against the MuSK protein varied between clones. A large number of wells with MuSK non-reactive clones derived from both the healthy donor cohort and MuSK MG patients were also tested. One clone from the healthy donor seemed to have some reactivity to MuSK in this assay (11-1B3).

FIG. 4 shows that the reactivity observed for MuSK clones isolated from MuSK MG patients were specific for MuSK, whereas the reactivity observed for the MuSK clone (11-1B3) that was isolated from a healthy donor was not. The specificity of MuSK reactivity was also tested for 4 MuSK MG clones using MuSK produced in yeast and compared to a control antigen (the acetylcholine receptor alpha subunit) produced in the same system. The “MuSK” positive clone from the healthy donor (11-1B3) did not show reactivity to MuSK produced in yeast suggesting that this antibody/BCR sequence recognized an E. Coli. related protein rather than being MuSK-specific. The MuSK clones isolated from MuSK MG patients (11-3F6, 11-7C5, 11-8G4, and 11-3D9) showed clear reactivity against yeast produced MuSK.

FIG. 5 shows sequence alignments using Clustal W alignment program.

FIG. 6—anti-MuSK/Anti-HIV-b12 exchanged heterospecific IgG4 antibodies were generated using methods detailed in Labrijn, 2014, Nature Protocols and Genmab, Labrijn, 2013, PNAS, which were adjusted to IgG4. These antibodies were tested for MuSK binding in mouse neuromuscular junction (NMJ) in ex vivo levator auris longus muscle. Equivalent anti-MuSK homospecific antibodies were also tested in parallel. The data confirms that both heterospecific MuSK IgG4 antibodies (13-3B5/HIV IgG4 and 11-3F6/HIV IgG4) and homospecific MuSK IgG4 antibodies (13-3B5 IgG4 and 11-3F6 IgG4) bind to MuSK at the NMJ.

FIG. 7—heterospecific MuSK IgG4 antibodies cause inhibition of agrin induced MuSK phosphorylation whereas homospecific MuSK IgG4 antibodies activate phosphorylation in absence of agrin.

FIG. 8 shows that homospecific versus heterospecific musk antibodies have differential effects on in vivo neuromuscular performance. Nod/scid mice were i.p. Injected with 5 ug/gbw recombinant antibody on day 0, 3 and 7. The heterospecific (functionally monovalent) version of both clones induce quick and severe muscle weakness and body weight loss. Homospecific 11-3F6 IgG4 did not induce muscle weakness or body weight loss. I.p.=intraperitoneal, gbw=gram body weight.

### DETAILED DESCRIPTION

The inventors have made recombinant IgG1 and IgG4 antibodies that are bivalent and monospecific for the Ig-like 1 domain of MuSK, using the variable domains of MuSK antibodies obtained from MuSK MG patients.

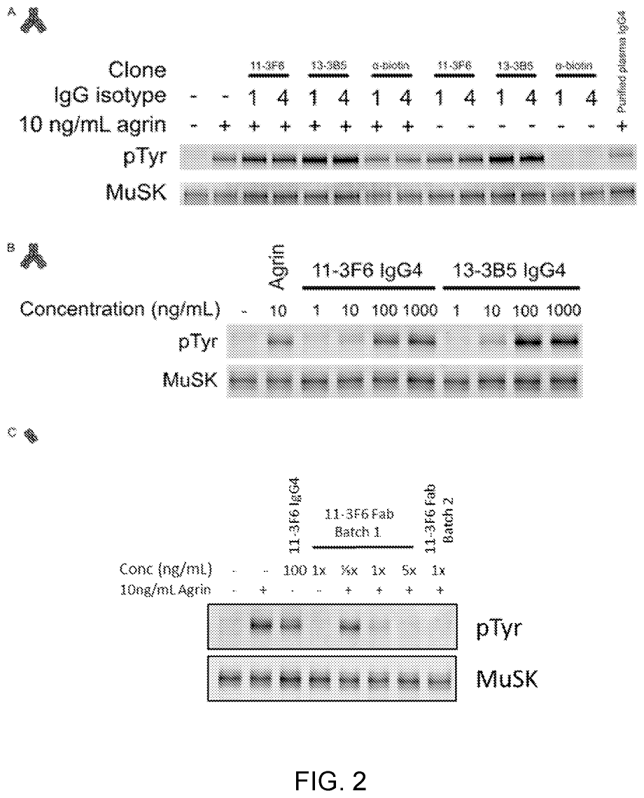

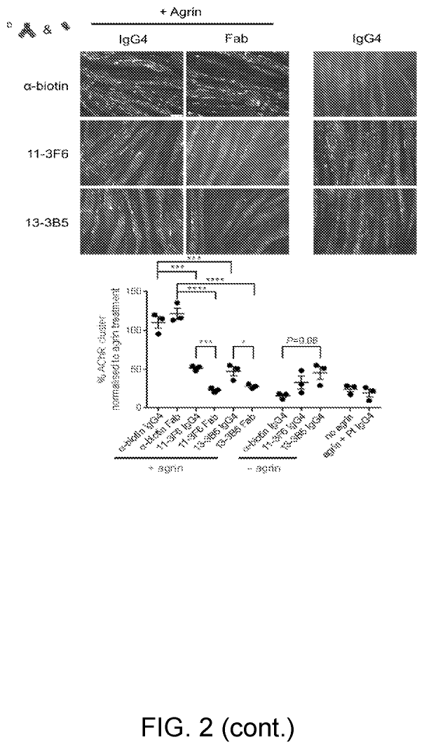

Surprisingly, patient-derived recombinant MuSK monoclonal antibodies (both IgG1 and IgG4) activated rather than inhibited MuSK phosphorylation (FIG. 2A). This effect was observed in both the absence and presence of agrin. Activation of MuSK phosphorylation was concentration-dependent (FIG. 2B) and differed slightly between the two clones. This suggests that patient-derived bivalent monospecific MuSK antibodies binding the Ig-like 1 domain facilitate dimerization and activation of MuSK in vitro.

Recombinant monoclonal IgG1 and IgG4 however both engage in bivalent monospecific antibody-antigen interactions. To investigate the functional effects of the bispecificity and functional monovalency of Fab-arm exchanged IgG4 MuSK antibodies in patients, the inventors generated monovalent Fab fragments from these recombinant antibodies by papain digestion. In vitro, these Fab fragments inhibited agrin-dependent MuSK phosphorylation (FIG. 2C) and AChR clustering similar to patient serum-derived anti-MuSK IgG4 (FIG. 2D). In contrast, (and in line with activating MuSK phosphorylation in vitro), bivalent monospecific monoclonal MuSK antibodies activated agrin-dependent AChR clustering compared to Fab fragments from the same monoclonal. Furthermore, AChR clustering could be partially induced using bivalent monospecific antibodies independent from agrin (FIG. 2D). Thus, monovalent MuSK binding blocks the AChR clustering pathway, whereas bivalent monospecific MuSK antibodies stimulate MuSK, and can facilitate or induce AChR clustering in this tissue culture model.

AChR clustering at the NMJ is critical for successful neuromuscular transmission and muscle contraction (Burden et al., 2018). Lower levels of AChR clustering are tolerated in patients until they reach a critical threshold. For example, for MuSK MG there is a dose dependent decrease in AChR clustering in mice passively transferred with MuSK antibodies (Klooster et al., 2012). Increasing AChR clustering will therefore result in improved neuromuscular transmission. Small increases in neuromuscular transmission can be therapeutically effective in patients (as exemplified by, for example, acetylcholine esterase treatment which is the first line symptomatic treatment for AChR MG patients). Advantageously, the bivalent monospecific MuSK antibodies described herein may therefore be used as a therapeutic agent to improve AChR clustering and synaptic stability at the NMJ.

The invention has been exemplified using antibodies. However, the general concept also applies to other binding agents, with binding regions that are specific for the MuSK Ig-like 1 domain. As an example, the data provided herein demonstrate that bivalent monospecific MuSK Ig-like 1 binding agents (i.e. binding agents with two binding regions, both of which are specific for the MuSK Ig-like 1 domain) can act as MuSK agonists (by inducing MuSK dimerization and/or phosphorylation and/or activation), whereas bivalent bi-specific MuSK Ig-like 1 binding agents (i.e. binding agents with two binding regions, only one of which is specific for the MuSK Ig-like 1 domain) can act as MuSK antagonists (by preventing MuSK dimerization and/or phosphorylation and/or activation).

The invention described herein is based on the finding that a binding agent comprising one or more binding regions, wherein only one binding region specifically binds to an Ig-like 1 domain of a MuSK protein can be used as a MuSK antagonist at the NMJ by inhibiting MuSK phosphorylation. A reduction in MuSK phosphorylation impairs neuromuscular transmission. The binding agents described herein may therefore be used to induce temporary or long term localised muscle weakness (paresis) in a subject. Advantageously, the binding agents described herein may therefore be used as an alternative to botulinum toxin therapies.

The binding agents described herein have a number of advantages over other known neuromuscular transmission antagonists such as botulinum toxin:
botulinum toxin is a multiprotein biological product that can vary in composition from one batch to another, which makes its activity less predictable or reproducible. It is estimated that approximately ˜400,000 mice per year are used in the EU alone to test the potency of each new batch of Botox (Taylor et al., 2019, ALTEX 36(1), 81-90). The binding agents described herein may be generated as e.g. monoclonal antibodies under stringent GCLP requirements, without any (or minimal) variation between batches. Advantageously, the binding agents described herein would therefore not require the same rigorous batch-to-batch testing as botulinum toxin.
botulinum toxin acts at the presynaptic nerve side and is also transported by retrograde axonal transport to the central nervous system, which might result in undesired side effects.
the binding agents described herein target the postsynaptic muscle membrane, are not likely to be internalized by the motor neuron so should not cause harm to the presynaptic nerve.
the binding agents described herein may be useful alternatives for subjects that have developed (or are at risk of developing) an allergic reaction to botulinum toxin.
the binding agents described herein may be useful alternatives for subjects that have developed (or are at risk of developing) an immune reaction to botulinum toxin.
the binding agents e.g. binding proteins such as antibodies described herein may be developed in an IgG4 background. Such binding agents cannot activate complement, thus do not cause any membrane damage and do not cause local inflammation.
the size of a binding agent described herein can be varied and made into a version that has favourable kinetics. For example, scFv can be used to access the target site more effectively, or multimer IgM-like molecules may be generated that have a longer half-life.

Binding Agents

A binding agent comprising one or more binding regions, wherein only one binding region specifically binds to an Ig-like 1 domain of a MuSK protein is described herein.

Advantageously, the binding agent inhibits MuSK activity by inhibiting MuSK phosphorylation and/or dimerization (which in turn inhibits the function of MuSK). In other words, the binding agents described herein do not (e.g. are not capable of) inducing MuSK phosphorylation and/or dimerization. The binding agent can be used to inhibit neuromuscular transmission in a subject.

The binding agent is therefore useful for treating a condition, disorder and/or symptom which is alleviated by the inhibition of neuromuscular transmission in a subject.

As defined in more detail below, “inhibit” or “inhibition” refers to a reduction or decrease and thus encompasses partial inhibition (e.g. inhibition of some but not all MuSK dimerization, and/or phosphorylation, and/or activation in a subject; and inhibition of some, but not all neuromuscular transmission in a subject).

In one specific example, the binding agent is a binding protein comprising one or more binding regions, wherein only one binding region specifically binds to an Ig-like 1 domain of a MuSK protein. In this context, the binding protein may be an antibody as described in more detail below, wherein the binding regions are variable regions. In this example, the antibody may comprise one or more variable regions, wherein only one variable region specifically binds to an Ig-like 1 domain of a MuSK protein.

The binding agent (e.g. binding protein, e.g. antibody) may be monovalent or multivalent. As used herein, a “monovalent” binding agent has only one binding region (also called an antigen-binding site herein). As used herein, a “multivalent binding agent” refers to a binding agent with a plurality of (i.e. more than one) binding regions (i.e. a plurality of antigen-binding sites).

A multivalent binding agent may therefore have two, three, four, five, six or more binding regions/antigen-binding sites. As an example a “bivalent” binding agent is a multivalent binding protein having two binding regions/antigen-binding sites, whereas a “trivalent” binding protein is a multivalent binding protein having three binding regions/antigen-binding sites.

The terms “binding region”, “antigen-binding site” and “epitope-binding site” are used interchangeably herein unless the context indicates otherwise.

As an example, the binding agent may have only one binding region (i.e. a binding region that specifically binds to an Ig-like 1 domain of a MuSK protein), and no other binding regions. In this context, the binding agent is monovalent.

For binding agents (e.g. binding proteins, e.g. antibodies) described herein that are multivalent, the binding agent (e.g. binding proteins, e.g. antibodies) only has one binding region that specifically binds to an Ig-like 1 domain of a MuSK protein. In other words, the remaining binding region(s) of the multivalent binding agent (i.e. the second, third, fourth, fifth etc. binding region of the multivalent binding agent) do not specifically bind to an Ig-like 1 domain of a MuSK protein; they specifically bind to a different antigen/epitope. Typically, the remaining binding region(s) of the multivalent binding agent (i.e. the second, third, fourth, fifth etc. binding region of the multivalent binding agent) specifically bind to a different antigen (e.g. they do not specifically bind to MuSK). However, for the avoidance of doubt, in an alternative example, the remaining binding region(s) of the multivalent binding agent (i.e. the second, third, fourth, fifth etc. binding region of the multivalent binding agent) may specifically bind to another region of the MuSK protein (e.g. the Ig-like 2 domain, Ig-like 3 domain, Frizzled domain etc.) provided that the multivalent binding agent does not (e.g. is not capable of) induce MuSK phosphorylation and/or dimerization and/or activation. Methods for identifying whether or not MuSK activation and/or dimerization and/or phosphorylation occurs are well known in the art.

As an example, the binding agent may have two binding regions (i.e. one binding region that specifically binds to an Ig-like 1 domain of a MuSK protein, and another binding region that is specific for another antigen/epitope (i.e. it does not specifically bind to the Ig-like 1 domain of the MuSK protein)). In this context, the binding agent may be bivalent (and bi-specific). This type of binding agent may also be termed “functionally monovalent” for the Ig-like 1 domain of a MuSK protein (i.e. it can only bind to Ig-like 1 domain of a MuSK protein via one binding region).

As a further example, the binding agent may have three binding regions (i.e. one binding region that specifically binds to an Ig-like 1 domain of a MuSK protein, and another two binding regions that are specific for another (one or two) antigen/epitopes (i.e. neither of the additional two binding regions specifically bind to the Ig-like 1 domain of the MuSK protein)). In this context, the binding protein may be trivalent (and bi-specific or tri-specific). As previously, this type of binding agent may also be termed “functionally monovalent” for the Ig-like 1 domain of a MuSK protein (i.e. it can only bind to Ig-like 1 domain of a MuSK protein via one binding region).

In one particular example, the binding agents described herein are antibodies. In this context, the binding region of the antibody may be a variable region.

The term “variable region” refers to the region of an immunoglobulin that comprises one or more Ig domains substantially encoded by any of the V_{κ}, Vλ, and/or VH genes that make up the kappa, lambda, and heavy chain immunoglobulin genetic loci respectively. More specifically, the term refers to the domain of an antibody heavy or light chain that is involved in binding the antibody to an antigen. The variable region of an immunoglobulin is therefore typically made up of two variable domains (i.e. the variable domain of the heavy chain and the variable domain of the light chain). The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007)). A single VH or VL domain may be sufficient to confer antigen-binding specificity.

The term “hypervariable region” refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) of the light chain variable domain and 31-35 (H1), 50-65 (H2) and 95-102 (H3) of the heavy chain variable domain according to Kabat et al, Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain according to Chothia and Lesk J. Mol. Biol. 196:901-917 (1987)).

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer. Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

An antibody of the invention may comprise one or more binding regions (i.e. variable regions), wherein only one binding region (i.e. variable region) specifically binds to an Ig-like 1 domain of a MuSK protein, In the context of an antibody, the terms variable region and binding region are used interchangeably herein. Each variable region may comprise the CDRs of a light chain variable domain (VL) (i.e. VL CDR1, VL CDR2, and VL CDR3) and/or the CDRs of a heavy chain variable domain (VH) (i.e. VH CDR1, VH CDR2, and VH CDR3). As stated elsewhere herein, the CDRs from one of VL or VH may be sufficient to confer antigen binding specificity (i.e. specific binding to the Ig-like 1 domain of MuSK). In other cases, antigen binding specificity (i.e. specific binding to the Ig-like 1 domain of MuSK) may be obtained by the presence of CDRs 1, 2, and 3, from both VL and VH.

Specific examples of combinations of CDRs that confer antigen binding specificity to the Ig-like 1 domain of MuSK are provided below. The CDR sequences have been identified using IMGT/V-QUEST program version: 3.4.17 (19 Feb. 2019)—IMGT/V-QUEST reference directory release: 201910-2 (5 Mar. 2019) (http://imgt.org/IMGT_vquest/vquest), selecting for Homo sapiens sequences. These CDR combinations form a binding region (i.e. variable region) of an antibody (including a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody) that specifically binds to an Ig-like 1 domain of a MuSK protein:

1) CDR1 comprising GFNFSTYT (SEQ ID NO:10), CDR2 comprising ISSRSAYK (SEQ ID NO: 11) and CDR3 comprising ARDFFQLGPPRFDS (SEQ ID NO:12). These CDRs may optionally be in the context of a VH, e.g. a VH comprising the sequence of SEQ ID NO:9.

2) CDR1 comprising QRISSF (SEQ ID NO:14), CDR2 comprising GAS (SEQ ID NO: 15) and CDR3 comprising QQSYSPMYT (SEQ ID NO:16). These CDRs may optionally be in the context of a VL, e.g. a VL comprising the sequence of SEQ ID NO:13.

3) CDRs of SEQ ID NO: 10, 11 and 12 (heavy chain) and CDRs of SEQ ID NO: 14, 15 and 16 (light chain) e.g. in the context of a VH and VL. For example, antigen specificity may be obtained by a combination of variable domains of SEQ ID NO: 9 (heavy chain) and SEQ: 13 (light chain).

4) CDR1 comprising GFTFSSYT (SEQ ID NO:18), CDR2 comprising IGSNGDYI (SEQ ID NO: 19) and CDR3 comprising ARGQLAVAGTHFDY (SEQ ID NO:20). These CDRs may optionally be in the context of a VH e.g. a VH comprising the sequence of SEQ ID NO:17.

5) CDR1 comprising QKVNKY (SEQ ID NO:22), CDR2 comprising AAS (SEQ ID NO: 23) and CDR3 comprising QQSYSPLCT (SEQ ID NO:24). These CDRs may optionally be in the context of a VL, e.g. a VL comprising the sequence of SEQ ID NO:21.

6) CDRs of SEQ ID NO: 18, 19 and 20 (heavy chain) and CDRs of SEQ ID NO: NO: 22, 23 and 24 (light chain) e.g. in the context of a VH and VL. For example, antigen specificity may be obtained by a combination of variable domains of SEQ ID NO:17 (heavy chain) and SEQ: 21 (light chain).

7) CDR1 comprising GFTFSDFT (SEQ ID NO:26), CDR2 comprising IGSSGTFI (SEQ ID NO: 27) and CDR3 comprising ARGRIAVAGTHFDL (SEQ ID NO:28). These CDRs may optionally be in the context of a VH, e.g. a VH comprising the sequence of SEQ ID NO:25.

8) CDR1 comprising GYTFTGQY (SEQ ID NO:30), CDR2 comprising INPSSGVT (SEQ ID NO: 31) and CDR3 comprising ATLSLGVYYVGMVA (SEQ ID NO:32). These CDRs may optionally be in the context of a VH, e.g. a VH comprising the sequence of SEQ ID NO:29.

9) CDR1 comprising GLAQQH (SEQ ID NO:34), CDR2 comprising KDI (SEQ ID NO: 35) and CDR3 comprising QSGDRTATSVL (SEQ ID NO:36). These CDRs may optionally be in the context of a VL, e.g. a VL comprising the sequence of SEQ ID NO:33.

10) CDRs of SEQ ID NO: 30, 31 and 32 (heavy chain) and CDRs of SEQ ID NO: 34, 35 and 36 (light chain) e.g. in the context of a VH and VL. For example, antigen specificity may be obtained by a combination of variable domains of SEQ ID NO: 29 (heavy chain) and SEQ: 33 (light chain).

11) CDR1 comprising GFDFSAST (SEQ ID NO:38), CDR2 comprising VSGDSHHI (SEQ ID NO: 39) and CDR3 comprising ARERLLRLGVGFDS (SEQ ID NO:40). These CDRs may optionally be in the context of a VH, e.g. a VH comprising the sequence of SEQ ID NO:37.

12) CDR1 comprising QRISGF (SEQ ID NO:42), CDR2 comprising AAS (SEQ ID NO: 43) and CDR3 comprising QQSYSPLYT (SEQ ID NO:44). These CDRs may optionally be in the context of a VL, e.g. a VL comprising the sequence of SEQ ID NO:41.

13) CDRs of SEQ ID NO: 38, 39 and 40 (heavy chain) and CDRs of SEQ ID NO: 42, 43 and 44 (light chain) e.g. in the context of a VH and VL. For example, antigen specificity may be obtained by a combination of variable domains of SEQ ID NO: 37 (heavy chain) and SEQ: 41 (light chain).

14) CDRs may be as follows: CDR1 comprising GFTFSSYT (SEQ ID NO:46), CDR2 comprising ISSGGHYI (SEQ ID NO: 47) and CDR3 comprising ARERLLRLGVGFDF (SEQ ID NO:48). These CDRs may optionally be in the context of a VH., e.g. a VH comprising the sequence of SEQ ID NO:45.

15) CDR1 comprising QSISGY (SEQ ID NO:50), CDR2 comprising AAS (SEQ ID NO: 51) and CDR3 comprising QQSYSALYT (SEQ ID NO:52). These CDRs may optionally be in the context of a VL, e.g. a VL comprising the sequence of SEQ ID NO:49.

16) CDRs of SEQ ID NO: 46, 47 and 48 (heavy chain) and CDRs of SEQ ID NO: 50, 51 and 52 (light chain) e.g. in the context of a VH and VL. For example, antigen specificity may be obtained by a combination of variable domains of SEQ ID NO: 45 (heavy chain) and SEQ: 49 (light chain).

17) CDR1 comprising GFTFSSYW (SEQ ID NO:54), CDR2 comprising LNEDGSTT (SEQ ID NO: 55) and CDR3 comprising VSDLSGKDEH (SEQ ID NO:56). These CDRs may optionally be in the context of a VH, e.g. a VH comprising the sequence of SEQ ID NO:53.

18) CDR1 comprising QSLLHSNGYYW (SEQ ID NO:58), CDR2 comprising LGF (SEQ ID NO: 59) and CDR3 comprising MQGLQ TPYT (SEQ ID NO:60). These CDRs may optionally be in the context of a VL, e.g. a VL comprising the sequence of SEQ ID NO:57.

19) CDRs of SEQ ID NO: 54, 55 and 56 (heavy chain) and CDRs of SEQ ID NO: 58, 59 and 60 (light chain) e.g. in the context of a VH and VL. For example, antigen specificity may be obtained by a combination of variable domains of SEQ ID NO: 53 (heavy chain) and SEQ: 57 (light chain).

The above CDRs may be in the context of an antibody, e.g. an IgG. For example, the IgG may be selected from IgG1, IgG2, IgG3, or IgG4.

For example, the CDR and variable domain sequences provided in options 1, 2, 3, 7, 11, 12, or 13 above may be present in the context of an IgG1 antibody Fc sequence. An exemplary sequence may be that of SEQ ID NO: 61 or a variant thereof (see for example the Fc region of SEQ ID NO: 65, 66 or 67).

For example, the CDR and variable domain sequences provided in options 8, 9 and 10 above may be present in the context of an IgG4 antibody Fc sequence. An exemplary sequence may be that of SEQ ID NO: 64 or a variant thereof (see for example the Fc region of SEQ ID NO: 69).

For example, the CDR and variable domain sequences provided in options 14, 15 or 16 above may be present in the context of an IgG3 antibody Fc sequence. An exemplary sequence may be that of SEQ ID NO: 63 or a variant thereof (see for example the Fc region of SEQ ID NO: 68).

As described in detail above, antibodies described herein (including a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody) comprise one or more binding regions, wherein only one binding region specifically binds to an Ig-like 1 domain of a MuSK protein.

In one embodiment, a binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:9; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:13. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:9 and comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:13 and comprises at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region comprises a heavy chain variable domain of SEQ ID NO:9 and a light chain variable domain of SEQ ID NO:13. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 9; and a light chain variable domain comprising a polypeptide sequence having at least 1, 2, 3, 4 or 5 conservative substitutions compared to a polypeptide sequence of SEQ ID NO: 13. Preferably, the binding region that specifically binds to the Ig-like 1 domain of a MuSK protein comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12. Preferably, the binding region that specifically binds to the Ig-like 1 domain of a MuSK protein comprises at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the binding region that specifically binds to the Ig-like 1 domain of a MuSK protein comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12 and at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 9; and a light chain variable domain of SEQ ID NO:13. Preferably, the binding region that specifically binds to the Ig-like 1 domain of a MuSK protein comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 9; and a light chain variable domain comprising a polypeptide sequence having at least 1, 2, 3, 4 or 5 conservative substitutions compared to a polypeptide sequence of SEQ ID NO: 13. Preferably, the binding region that specifically binds to the Ig-like 1 domain of a MuSK protein comprises at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:10, a VH CDR2 having SEQ ID NO:11 and a VH CDR3 having SEQ ID NO:12 and at least one, particularly at least two, more particularly at least 3 of the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:10, a VH CDR2 having SEQ ID NO:11 and a VH CDR3 having SEQ ID NO:12 and the light chain CDR's: a VL CDR1 having SEQ ID NO:14, a VL CDR2 having SEQ ID NO:15 and a VL CDR3 having SEQ ID NO:16. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment an antibody binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:17; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:21. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:17 and comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:21 and comprises at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:17 and a light chain variable domain of SEQ ID NO:21. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 17; and a light chain variable domain comprising a polypeptide sequence having at least 1, 2, 3, 4 or 5 conservative substitutions compared to a polypeptide sequence of SEQ ID NO: 21. Preferably, the binding region that specifically binds to the Ig-like 1 domain of a MuSK protein comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20. Preferably, the binding region that specifically binds to the Ig-like 1 domain of a MuSK protein comprises at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the binding region that specifically binds to the Ig-like 1 domain of a MuSK protein comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20 and at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 17; and a light chain variable domain of SEQ ID NO:21. Preferably, the binding region that specifically binds to the Ig-like 1 domain of a MuSK protein comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 17; and a light chain variable domain comprising a polypeptide sequence having at least 1, 2, 3, 4 or 5 conservative substitutions compared to a polypeptide sequence of SEQ ID NO: 21. Preferably, the binding region that specifically binds to the Ig-like 1 domain of a MuSK protein comprises at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:18, a VH CDR2 having SEQ ID NO:19 and a VH CDR3 having SEQ ID NO:20 and at least one, particularly at least two, more particularly at least 3 of the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:18, a VH CDR2 having SEQ ID NO:19 and a VH CDR3 having SEQ ID NO:20 and the light chain CDR's: a VL CDR1 having SEQ ID NO:22, a VL CDR2 having SEQ ID NO:23 and a VL CDR3 having SEQ ID NO:24. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:25. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:25 and comprises at least one, particularly at least two, more particularly at least 3 of the heavy chain CDR's: a VH CDR1 having SEQ ID NO:26, a VH CDR2 having SEQ ID NO:27 and a VH CDR3 having SEQ ID NO:28. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:25. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 25. Preferably, the binding region that specifically binds to the Ig-like 1 domain of a MuSK protein comprises at least one, particularly at least two, more particularly at least 3 of the heavy chain CDR's: a VH CDR1 having SEQ ID NO:26, a VH CDR2 having SEQ ID NO:27 and a VH CDR3 having SEQ ID NO:28. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:26, a VH CDR2 having SEQ ID NO:27 and a VH CDR3 having SEQ ID NO:28. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:26, a VH CDR2 having SEQ ID NO:27 and a VH CDR3 having SEQ ID NO:28. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment the binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:29; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:33. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:29 and comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:33 and comprises at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:29 and a light chain variable domain of SEQ ID NO:33. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 29; and a light chain variable domain comprising a polypeptide sequence having at least 1, 2, 3, 4 or 5 conservative substitutions compared to a polypeptide sequence of SEQ ID NO: 33. Preferably, the binding region that specifically binds to the Ig-like 1 domain of a MuSK protein comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32. Preferably, the binding region that specifically binds to the Ig-like 1 domain of a MuSK protein comprises at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the binding region that specifically binds to the Ig-like 1 domain of a MuSK protein comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32 and at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 29; and a light chain variable domain of SEQ ID NO:33. Preferably, the binding region that specifically binds to the Ig-like 1 domain of a MuSK protein comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 29; and a light chain variable domain comprising a polypeptide sequence having at least 1, 2, 3, 4 or 5 conservative substitutions compared to a polypeptide sequence of SEQ ID NO: 33. Preferably, the binding region that specifically binds to the Ig-like 1 domain of a MuSK protein comprises at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:30, a VH CDR2 having SEQ ID NO:31 and a VH CDR3 having SEQ ID NO:32 and at least one, particularly at least two, more particularly at least 3 of the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:30, a VH CDR2 having SEQ ID NO:31 and a VH CDR3 having SEQ ID NO:32 and the light chain CDR's: a VL CDR1 having SEQ ID NO:34, a VL CDR2 having SEQ ID NO:35 and a VL CDR3 having SEQ ID NO:36. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:37; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:41. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:37 and comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:41 and comprises at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:37 and a light chain variable domain of SEQ ID NO:41. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 37; and a light chain variable domain comprising a polypeptide sequence having at least 1, 2, 3, 4 or 5 conservative substitutions compared to a polypeptide sequence of SEQ ID NO: 41. Preferably, the binding region that specifically binds to the Ig-like 1 domain of a MuSK protein comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40. Preferably, the binding region that specifically binds to the Ig-like 1 domain of a MuSK protein comprises at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the binding region that specifically binds to the Ig-like 1 domain of a MuSK protein comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40 and at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 37; and a light chain variable domain of SEQ ID NO:41. Preferably, the binding region that specifically binds to the Ig-like 1 domain of a MuSK protein comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 37; and a light chain variable domain comprising a polypeptide sequence having at least 1, 2, 3, 4 or 5 conservative substitutions compared to a polypeptide sequence of SEQ ID NO: 41. Preferably, the binding region that specifically binds to the Ig-like 1 domain of a MuSK protein comprises at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:38, a VH CDR2 having SEQ ID NO:39 and a VH CDR3 having SEQ ID NO:40 and at least one, particularly at least two, more particularly at least 3 of the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:38, a VH CDR2 having SEQ ID NO:39 and a VH CDR3 having SEQ ID NO:40 and the light chain CDR's: a VL CDR1 having SEQ ID NO:42, a VL CDR2 having SEQ ID NO:43 and a VL CDR3 having SEQ ID NO:44. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:45; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:49. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:45 and comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:49 and comprises at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:45 and a light chain variable domain of SEQ ID NO:49. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 45; and a light chain variable domain comprising a polypeptide sequence having at least 1, 2, 3, 4 or 5 conservative substitutions compared to a polypeptide sequence of SEQ ID NO: 49. Preferably, the binding region that specifically binds to the Ig-like 1 domain of a MuSK protein comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48. Preferably, the binding region that specifically binds to the Ig-like 1 domain of a MuSK protein comprises at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the binding region that specifically binds to the Ig-like 1 domain of a MuSK protein comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48 and at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 45; and a light chain variable domain of SEQ ID NO:49. Preferably, the binding region that specifically binds to the Ig-like 1 domain of a MuSK protein comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 45; and a light chain variable domain comprising a polypeptide sequence having at least 1, 2, 3, 4 or 5 conservative substitutions compared to a polypeptide sequence of SEQ ID NO: 49. Preferably, the binding region that specifically binds to the Ig-like 1 domain of a MuSK protein comprises at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:46, a VH CDR2 having SEQ ID NO:47 and a VH CDR3 having SEQ ID NO:48 and at least one, particularly at least two, more particularly at least 3 of the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:46, a VH CDR2 having SEQ ID NO:47 and a VH CDR3 having SEQ ID NO:48 and the light chain CDR's: a VL CDR1 having SEQ ID NO:50, a VL CDR2 having SEQ ID NO:51 and a VL CDR3 having SEQ ID NO:52. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises: a heavy chain variable domain having an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:53; and a light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:57. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Preferably, the heavy chain variable domain has an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:53 and comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO:56; and light chain variable domain an amino acid sequence that is 85%, 86%, 87%, 88%, 89%, 90%, 91%, 92%, 93%, 94%, 95%, 96%, 97%, 98%, 99% or 100% identical to the polypeptide of SEQ ID NO:57 and comprises at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

Most preferably, the binding region that specifically binds to the Ig-like 1 domain of a MuSK protein comprises a heavy chain variable domain of SEQ ID NO:53 and a light chain variable domain of SEQ ID NO:57. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 53; and a light chain variable domain comprising a polypeptide sequence having at least 1, 2, 3, 4 or 5 conservative substitutions compared to a polypeptide sequence of SEQ ID NO: 57. Preferably, the binding region that specifically binds to the Ig-like 1 domain of a MuSK protein comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO56. Preferably, the binding region that specifically binds to the Ig-like 1 domain of a MuSK protein comprises at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the binding region that specifically binds to the Ig-like 1 domain of a MuSK protein comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO:56 and at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain comprising a polypeptide sequence having at least 1, 2, 3, 4 or 5 conservative substitutions compared to a polypeptide sequence of SEQ ID NO: 53; and a light chain variable domain of SEQ ID NO:57. Preferably, the binding region that specifically binds to the Ig-like 1 domain of a MuSK protein comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO:56. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises a heavy chain variable domain of SEQ ID NO: 53; and a light chain variable domain comprising a polypeptide sequence having at least 1, 2, 3, 4 or 5 conservative substitutions compared to a polypeptide sequence of SEQ ID NO: 57. Preferably, the binding region that specifically binds to the Ig-like 1 domain of a MuSK protein comprises at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO:56. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises at least one, particularly at least two, more particularly at least 3 of the heavy chain CDR's: a VH CDR1 having SEQ ID NO:54, a VH CDR2 having SEQ ID NO:55 and a VH CDR3 having SEQ ID NO:56 and at least one, particularly at least two, more particularly at least 3 of the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

In one embodiment, a binding region that specifically binds to the Ig-like 1 domain of a MuSK protein comprises the heavy chain CDR's: a VH CDR1 having SEQ ID NO:54, a VH CDR2 having SEQ ID NO:55 and a VH CDR3 having SEQ ID NO:56 and the light chain CDR's: a VL CDR1 having SEQ ID NO:58, a VL CDR2 having SEQ ID NO:59 and a VL CDR3 having SEQ ID NO:60. Preferably, the antibody is selected from an antibody, a Fab, bi-specific Fab₂, tri-specific Fab₃ scFv, bi-specific di-scFv, bi-specific scFv-Fc, bi-specific diabody, a tri-specific triabody, a single domain antibody or a bi-specific minibody. The antibody comprises one or more binding regions, wherein only one binding region specifically binds to an Ig-like 1 domain of a MuSK protein (e.g. via the sequences set forth above).

As used herein, the terms “homology” and “identity” are used interchangeably. Calculations of sequence homology or identity between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

As used herein the term “conservative amino acid substitution” refers to replacement of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In the context of binding, the terms “specific” and “specifically” are used herein interchangeably to indicate that other biomolecules do not significantly bind to the binding region (e.g. CDR, variable region etc.) that is specifically binding to the biomolecule of interest (where the biomolecule of interest is the Ig-like 1 domain of a MuSK protein). In some examples, the level of binding to a biomolecule other than the Ig-like 1 domain of a MuSK protein results in a negligible (e.g., not determinable) binding affinity by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to an Ig-like 1 domain of a MuSK protein.

The binding affinity of a binding region with an Ig-like 1 domain of a MuSK protein may be determined using a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al. (1991) Biotechniques 11:620-627; Johnsson, B., et al (1995) J. Mol. Recognit. 8: 125-131; and Johnson, B., et al. (1991) Anal. Biochem. 198:268-277.

For example, “specifically binding” in the context of the binding of an antibody to a predetermined antigen/epitope means binding with an affinity corresponding to a KD (equilibrium dissociation constant) of about 10⁻⁷ M or less, such as about 10⁻⁸ M or less, such as about 10⁻⁹ M or less, about 10⁻¹⁰ M or less, or about 10⁻¹¹ M or even less when determined for instance by surface plasmon resonance (SPR) technology in a BIAcore 3000 instrument using the antibody as the analyte (wherein a low KD indicates a high affinity). This term also means that the antibody binds to the predetermined antigen/epitope with an affinity corresponding to a KD that is at least ten-fold lower, such as at least 100 fold lower, for instance at least 1000 fold lower, such as at least 10,000 fold lower, for instance at least 100,000 fold lower than its affinity for binding to a non-specific antigen (e.g. bovine serum albumin, casein) other than the predetermined antigen or a closely-related antigen. The amount with which the affinity is lower is dependent on the KD of the antibody, so that when the KD of the antibody is very low (that is, the antibody is very specific and binds very well), then the amount with which the affinity for the antigen is lower than the affinity for a non-specific antigen may be at least 10,000 fold. The term “KD” as used herein, means the dissociation rate constant of a particular antibody-antigen interaction.

Equivalent affinity values for “specific binding” of other binding agents described herein are well known in the art.

A binding region described herein may be specific for (i.e. specifically bind to) an epitope within the Ig-like 1 domain of a MuSK protein. As used herein the term “epitope” refers to a site on a target molecule (e.g., an antigen, such as a protein, for example an Ig-like 1 domain of a MuSK protein) to which a binding agent (e.g., a binding protein such as an antibody or antibody fragment) binds. Epitopes are groupings of molecules such as amino acids or sugar side chains and usually have specific structural characteristics, as well as specific charge characteristics. A single antigen may have more than one epitope. Epitopes can be formed both from contiguous or adjacent noncontiguous residues (e.g., amino acid residues) of the target molecule. Epitopes formed from contiguous residues (e.g., amino acid residues) typically are also called linear epitopes. An epitope typically includes at least 5 and up to about 12 residues, mostly between 6 and 10 residues (e.g. amino acid residues). Epitopes may also be conformational (i.e. non-linear).

In one example, the MuSK protein is a human MuSK protein. The MuSK protein in humans is well characterized. The MuSK protein has been sequenced and the protein characterized recently by Valenzuela et al. (see WO97/21811). It is a receptor tyrosine kinase (RTK) located on the cell surface of muscle cells at the NMJ and has the sequence shown in SEQ ID NO:1. The human MuSK protein can be identified by UniprotKB identifier: O15146. Human MuSK gene information can be found at Ensembl ref: ENSG00000030304. The gene has seven transcripts, as outlined below:

TABLE 1
The seven transcripts of human MuSK.

| Name | Transcript ID | bp | Protein | Translation ID | Biotype | CCDS | UniProt | RefSeq |
|---|---|---|---|---|---|---|---|---|
| MUSK-205 | ENST00000374448.8 | 2754 | 869 | ENSP00000363571 | Protein | CCDS | O15146 | NM_005592 |
| | | | aa | | coding | 48005 | | NP_005583 |
| MUSK-201 | ENST00000189978.10 | 2408 | 783 | ENSP00000189978 | Protein | CCDS | O15146 | NM_001166280 |
| | | | aa | | coding | 75874 | | NM_001166281 |
| | | | | | | | | NP_001159752 |
| | | | | | | | | NP_001159753 |
| MUSK-206 | ENST00000416899.7 | 3344 | 861 | ENSP00000393608 | Protein | — | A0A087WSY1 | — |
| | | | aa | | coding | | | |
| MUSK-204 | ENST00000374440.7 | 2408 | 783 | ENSP00000363563 | Protein | — | Q5T0B5 | — |
| | | | aa | | coding | | | |
| MUSK-203 | ENST00000374439.1 | 435 | 132 | ENSP00000363562 | Protein | — | F6XAJ2 | — |
| | | | aa | | coding | | | |
| MUSK-207 | ENST00000634612.1 | 541 | No | — | Processed | — | — | — |
| | | | protein | | transcript | | | |
| MUSK-202 | ENST00000374438.1 | 1221 | No | — | Retained | — | — | — |
| | | | protein | | intron | | | |

The extracellular region of MuSK contains three Ig-like domains and a Frizzled-like domain. The first N-terminal Ig-like domain (also known as the Ig-like 1 domain herein) in MuSK is required for MuSK to bind Lrp4 (Zhang et al., 2011). Mutation of a single residue, 196, on a solvent exposed surface of the first Ig-like 1 domain, prevents MuSK from binding Lrp4 and responding to agrin (Stiegler et al., 2006; Zhang et al., 2011). A hydrophobic surface on the opposite side of the first Ig-like 1 domain mediates MuSK homodimerization, essential for MuSK trans-phosphorylation. Although MuSK is expressed by muscle and not by motor neurons, MuSK is essential for presynaptic as well as postsynaptic differentiation (Burden et al., 2013). MuSK regulates presynaptic differentiation by clustering Lrp4 in muscle, which functions bi-directionally by serving not only as a receptor for agrin and a ligand for MuSK, but also as a direct retrograde signal for presynaptic differentiation. In addition to its role during synapse formation, MuSK is also required to maintain adult synapses, as inhibition of MuSK expression in adult muscle leads to profound defects in presynaptic and postsynaptic differentiation (Hesser et al., 2006).

As described herein, one binding region of the binding agent is specific for (i.e. specifically binds to) an epitope within the Ig-like 1 domain of a human MuSK protein (also referred to herein as the Immunoglobulin-like 1 domain of MuSK; the MuSK Ig-like 1 domain; or the first Ig-like 1 domain of MuSK etc.). The Ig-like 1 domain of the human MuSK protein has the sequence shown in SEQ ID NO:2: PVITTPLETVDALVEEVATFMCAVESYPQPEISWTRNKILIKLFDTRYSIRENGQLLTILSVEDS DDGIYCCTANNGVGGAVESCGALQV

As used herein, a “monospecific” binding agent therefore refers to a binding agent that contains one antigen-binding site only, or a plurality (e.g. two, three, four, five etc.) of antigen-binding sites (e.g. variable regions) each of which are identical (or at least each of which bind to the same epitope in the target antigen). In the context of binding agents that act as MuSK antagonists, as described herein, the binding agent (e.g. binding protein such as an antibody) may be monospecific if e.g. it only has one binding region, and that binding region specifically binds to the Ig-like 1 domain of a MuSK protein.

For binding proteins described herein with a plurality of binding regions, the binding agent is necessarily “multi-specific” (e.g. bi-specific, or tri-specific etc.). This is because the binding agents described herein may only have one binding region that is specific for the Ig-like 1 domain of a MuSK protein. A “bi-specific” binding agent therefore contains two antigen-binding sites that are different (and thus a bi-specific binding protein binds to two different antigens/epitopes).

The binding agent may be any appropriate binding agent known in the art. Non-limiting examples of binding agents described herein include, but are not limited to aptamers (e.g. nucleic acid aptamers, peptide aptamers, aptabodies, affimers), binding proteins (e.g. antibodies, antibody mimetics, camelid antibodies, duobodies etc.) and small molecules. Other appropriate binding agents are also well known and readily identifiable to a person of skill in the art using routine experimental procedures.

As used herein, “aptamer” refers to nucleic acid aptamers and/or peptide aptamers. Examples of aptamers include affimers (an evolution of peptide aptamers) and aptabodies (formed by hybridisation of two DNA aptamers), which are also well known and readily identifiable to a person of skill in the art using routine experimental procedures.

In one example, the binding agent is a binding protein. Examples of appropriate binding proteins include antibodies and antibody mimetics. Examples of antibodies are provided elsewhere herein. Examples of appropriate antibody mimetics include affibody molecules (including affimabs) affilins, peptide aptamers (including affimers), affitins, alphabodies, anticalins, avimers, DARPins, Fynomers, Kunitz domain peptides, monobodies, nanoCLAMPs etc., which are also well known and readily identifiable to a person of skill in the art using routine experimental procedures.

As stated elsewhere herein, and as shown in the examples, the binding agent may be an antibody. The terms “antibody” or “antibodies” as used herein refer to molecules or active fragments of molecules that bind to known antigens, particularly it refers to immunoglobulin molecules and to immunologically active portions of immunoglobulin molecules, i.e. molecules that contain a binding site that specifically binds an antigen. The immunoglobulin described herein can be of any class (IgG, IgM, IgD, IgE, IgA and IgY) or subclass (e.g. IgG1, IgG2, IgG3, IgG4, IgA1 and IgA2) of immunoglobulin molecule and based on heavy chain sequences from any species. For example, the species may be, but not limited to dogs, cats, horses, cows, pigs, guinea pigs, mice, rats and the like. The species may be a primate (e.g. a non-human primate). In a preferred example, the species is a human.

The term “antibody” or “antibodies” include monoclonal, polyclonal, chimeric, single chain, bi-specific, human and humanized antibodies as well as active fragments thereof. Examples of active fragments of molecules that bind to known antigens and are useful include Fab, bi-specific Fab₂, tri-specific Fab₃, scFv, bi-specific di-scFv, bi-specific scFv-Fc, bi-specific diabody, a duobody, a tri-specific triabody, a single domain antibody and a bi-specific minibody, including the products of an Fab immunoglobulin expression library and epitope-binding fragments of any of the antibodies and fragments mentioned above.

In a particular example, the antibody may be a monoclonal antibody. As used herein, the term “monoclonal antibody” refers to an antibody that can be mass produced in the laboratory from a single clone and that recognizes only one antigen. Monoclonal antibodies may be generated by any appropriate technique known in the art (e.g. by production in HEK or insect cells, or by generation of B cell hybridomas).

As used herein, the term “chimeric antibody” refers to a monoclonal antibody comprising a variable region, i.e., binding region, from one source or species, (i.e. non-human primates, humans, dogs, cats, horses, cows, pigs, guinea pigs, mice, rats and the like) and at least a portion of a constant region derived from a different source or species, usually prepared by recombinant DNA techniques. Chimeric antibodies comprising a mouse variable region and a human constant region are examples. Such chimeric antibodies are the product of expressed immunoglobulin genes comprising DNA segments encoding mouse immunoglobulin variable regions and DNA segments encoding human immunoglobulin constant regions. Other forms of “chimeric antibodies” encompassed by the present disclosure are those in which the class or subclass has been modified or changed from that of the original antibody, for example to alter them so that they are complement and Fc receptor binding deficient. Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. Nos. 5,202,238 and 5,204,244.

In a particular example, the antibodies described herein are complement and Fc receptor binding deficient.

In a particular example, the antibody may be a human antibody or a humanized antibody.

As used herein the term “humanized antibody” or “humanized version of an antibody” refers to antibodies in which the framework or “complementarity determining regions” (CDR) have been modified to comprise the CDR of an immunoglobulin of different specificity as compared to that of the parent immunoglobulin. In some examples, the CDRs of the VH and VL are grafted into the framework region of human antibody to prepare the “humanized antibody.” See e.g. Riechmann, L., et al., Nature 332 (1988) 323-327; and Neuberger, M. S., et al., Nature 314 (1985) 268-270. The heavy and light chain variable framework regions can be derived from the same or different human antibody sequences. Both the heavy chain and the light chain may be required for effective antigen (MuSK) binding. The human antibody sequences can be the sequences of naturally occurring human antibodies. Human heavy and light chain variable framework regions are listed e.g. in Lefranc, M.-P., Current Protocols in Immunology (2000)—Appendix 1P A.1P.1-A.1P.37 and are accessible via IMGT, the international ImMunoGeneTics information System® (http://imgt.cines.fr) or via http://vbase.mrc-cpe.cam.ac.uk, for example. Optionally the framework region can be modified by further mutations. Exemplary CDRs correspond to those representing sequences recognizing the antigens noted above for chimeric antibodies. In some examples, such humanized version is chimerized with a human constant region.

As used herein the term “human antibody” is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice results in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. R. (1985) p. 77; and Boerner, P., et al., J. Immunol. 147 (1991) 86-95).

In a particular example, the antibody may be selected from a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody.

Other examples include single domain antibodies such as those found in camelids including, but not limited to, llamas and alpacas; and cartilaginous fish including, but not limited to, sharks which are widely known in the art.

As used herein “single chain antibody” refers to single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883 or a bi-specific single chain Fv (WO 03/11161). Typical scFv linkers are well known in the art, are generally 10 to 25 amino acids in length and include glycines and serines.

Single domain antibody fragments (VHH or nanobodies) e.g. the functional antibodies produced by camelids that are devoid of light chains and wherein a single N-terminal domain is fully capable of antigen binding are also an example of a binding agent described herein. Such fragments can also form bivalent VHH, or pentabodies (i.e. with 5 VHH domains).

As used herein, a “di-ScFv” refers to a dimerized scFV.

As used herein, “minibodies” are minimized antibody-like proteins comprising a scFv joined to a CH3 domain. See Hu et al., 1996, Cancer Res. 56:3055-3061. In some cases, the scFv can be joined to the Fc region, and may include some or the entire hinge region.

By “Fab” or “Fab region” as used herein is meant the polypeptides that comprise the VH, CH1, VH, and CL immunoglobulin domains. Fab may refer to this region in isolation, or this region in the context of a full length antibody or antibody fragment or fab fusion protein.

The terms “Fab”, “Fab region”, “Fab portion” or “Fab fragment” are understood to define a polypeptide that includes a VH, a CH1, a VL, and a CL immunoglobulin domain. Fab may refer to this region in isolation, or this region in the context of an antibody molecule described herein, as well as a full length immunoglobulin or immunoglobulin fragment. Typically a Fab region contains an entire light chain of an antibody. A Fab region can be taken to define “an arm” of an immunoglobulin molecule. It contains the epitope-binding portion of that Ig. The Fab region of a naturally occurring immunoglobulin can be obtained as a proteolytic fragment by a papain-digestion. A “F(ab′)2 portion” is the proteolytic fragment of a pepsin-digested immunoglobulin. A “Fab′ portion” is the product resulting from reducing the disulfide bonds of an F(ab′)2 portion. As used herein the terms “Fab”, “Fab region”, “Fab portion” or “Fab fragment” may further include a hinge region that defines the C-terminal end of the antibody arm (cf. above). This hinge region corresponds to the hinge region found C-terminally of the CH1 domain within a full length immunoglobulin at which the arms of the antibody molecule can be taken to define a Y. The term hinge region is used in the art because an immunoglobulin has some flexibility at this region.

By “Fc fusion” as used herein is meant a protein wherein one or more polypeptides is operably linked to Fc. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200). An Fc fusion combines the Fc region of an immunoglobulin with a fusion partner, which in general may be any protein, polypeptide or small molecule. The role of the non-Fc part of an Fc fusion, i.e., the fusion partner, is to mediate target binding, and thus it is functionally analogous to the variable regions of an antibody. Virtually any protein or small molecule may be linked to Fc to generate an Fc fusion. Protein fusion partners may include, but are not limited to, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, e.g., an extracellular receptor that is implicated in disease.

As used herein the term “antibody fragments” refers to a portion of a full length antibody, for example possibly a variable domain thereof, or at least an antigen binding site thereof. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. Antibody fragments can be derived from an antibody described herein by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

The binding proteins provided herein may be encoded by a nucleic acid molecule that has been codon optimized to increase protein expression in the appropriate host (e.g. HEK cells or insect cells). Codon optimization and protein production techniques are well known in the art and can readily be adapted for the production of binding proteins described herein.

In a particular example, the binding protein described herein may be an IgG. The IgG may be selected from IgG1, IgG2 or IgG3. In an alternative example, the binding protein described herein may be an IgG4 variant (described in more detail below).

In vivo, IgG4 naturally undergoes Fab-arm exchange which renders it functionally bi-specific preventing crosslinking and internalization of the antigen (van der Neut Kolfschoten et al., 2007). The term “Fab-arm exchange” or “FAE” as used herein means the exchange and recombination of one complete heavy-light chain pair (half-molecule or half-IgG) of one antibody with that of another antibody. In other words, “Fab-arm exchange” is used herein (and in the wider literature) to refer to the process leading to the exchange of human IgG4 “half molecules” (half IgG exchange—see for example: Schuurman et al., mAbs 4:6, 636-636 November/December 2012). Fab-arm exchange is also known as IgG4 shuffling, in which increased susceptibility of native IgG4 hinge disulfide bonds to reduction allows the heavy chains to separate and randomly re-associate to produce a mixed population of IgG4 molecules with randomized heavy-chain and light-chain pairs (Aalberse et al., 1999. Int Arch Allergy Immunol 118: 187-189; Labrijn, et al, 2009, Nat Biotechnol 27:767-771; Schuurman et al, 2001. Mol Immunol 38: 1-8; van der Neut Kolfschoten et al, 2007. Science 317: 1554-1557). See also Makaraviciute A, Jackson C D, Millner P A, Ramanaviciene A. J Immunol Methods. 2016 February; 429:50-6. The terms “Fab-arm exchange”, “half-antibody exchange” and “half-IgG exchange” are therefore used interchangeably herein.

In the context of the invention, and in the context of a binding protein with multiple variable domains (e.g. a bi-specific binding protein, having only one variable region that is specific for an Ig-like 1 domain of a MuSK protein) it is advantageous to reduce or remove the ability of an IgG4 to undergo Fab-arm exchange (i.e. to ensure that the binding protein retains only one variable region that specifically binds to an Ig-like 1 domain of a MuSK protein, and thus retains its MuSK antagonist properties), or to use introduce the binding region that specifically binds to the Ig-like 1 domain of a MuSK protein into an IgG1 (or IgG2, or IgG3) background, wherein the resultant IgG is bi-specific (i.e. only has one binding region that specifically binds to the Ig-like 1 domain of a MuSK protein as described elsewhere herein).

The binding protein described herein may therefore be an IgG4 variant with reduced ability for Fab-arm exchange in vivo (e.g. the IgG4 may be incapable of undergoing Fab-arm exchange). This type of binding protein is particularly useful as IgG4 antibodies are considered as “anti-inflammatory” antibodies because they are unable to bind complement or recruit immune cells (Lighaam et al., 2016). Accordingly, IgG4 variants described herein are less likely to induce an inflammatory response when administered to a subject as described herein (compared to an equivalent IgG1, IgG2 or IgG3).

An IgG4 antibody variant with reduced ability for Fab-arm exchange in vivo may therefore be generated for use in the methods described herein, wherein the IgG4 variant comprises two variable regions, wherein only one of the variable regions in the binding protein specifically binds to an Ig-like 1 domain of a MuSK protein.

As used herein, an IgG4 variant with “reduced ability for Fab-arm exchange in vivo” refers to an IgG4 that has a lower rate of Fab-arm exchange than a reference IgG4 antibody, under the same (in vivo) conditions. A “reduced ability” refers to a reduction in the Fab-arm exchange rate (e.g. at least a 5%, at least 10%, at least 15%, at least 20% at least 30%, at least 40%, at least 50%, at least 60%, at least 70%, at least 80%, at least 85%, at least 90%, at least 95% reduction in the rate of Fab-arm exchange over a defined time period (set from the point of the start of the test, e.g. administration of the IgG4 to a subject like a mouse or human subject)). Methods for measuring the rate of Fab-arm exchange are known in the art.

A reduction in IgG4 shuffling/Fab-arm exchange may be determined by detecting of a lower amount of half antibody molecules, detection of new bispecific antibodies or of arm exchange produced from an IgG4 variant described herein (which e.g. contains one or more amino acid substitutions in the hinge or CH3 region), as compared to the amount of half antibody molecules or of arm exchange produced from an IgG4 molecule containing an IgG4 hinge or CH3 region not comprising said one or more amino acid substitutions. Any assay well-known in the art can be used to detect half antibody production and bi-specific antibody molecules. See, e.g., Van der Neut Kolfschoten et al, 2007, Science, 317: 1554-1557, for examples of assays to detect production of bi-specific antibodies.

As used herein, an IgG4 variant that is “incapable of undergoing Fab-arm exchange” means that the IgG4 variant is not able to exchange and recombine one complete heavy-light chain pair (half-molecule) of one antibody with that of another antibody in vivo (e.g. when measured over a 24 hour period set from the point of administration of the IgG4 to a subject e.g. a mouse subject or a human subject) or in vitro (e.g. when using a reducing agent such as glutathione). In other words, in this context an IgG4 variant that is bivalent and bi-specific (i.e. one arm binding specifically to the Ig-like 1 domain of a MuSK protein, and one arm binding to an unrelated protein) would not be capable of Fab-arm exchange and thus would remain bi-specific in vivo (e.g. when measured over a defined time period set from the point of start of the test, e.g. administration of the IgG4 to a subject like a mouse subject or a human subject). Methods for determining whether an IgG4 undergoes Fab-arm exchange are known in the art and are described above.

In one example, the IgG4 variant comprises an IgG4 constant region comprising one or more amino acid substitutions that reduce the ability for Fab-arm exchange in vivo.

For example, the IgG4 variant is incapable of undergoing Fab-arm exchange because it comprises an IgG4 constant region comprising an amino acid substitution at amino acid position 228 and/or an amino acid substitution at amino acid position 405 and/or an amino acid substitution at amino acid position 409 of the heavy chain numbered according to the EU index (See, Edelman et al., 1969, Proc. Natl. Acad. Sci. USA, 63(1): 78-85 and Huijbers 2016). One or more of these substitutions in the hinge or CH3 region of human IgG4 results in considerable reduction of intra-chain disulfide bond formation, resulting in the reduction of IgG4 “half-antibody” molecules and reduced heterogeneity/shuffling of IgG4 molecules (Bloom et al. 1997, Protein Sci, 6:407-415; Angal et al, 1993, Molecular Immunology, 30(1): 105-108)). There are also published reports that these hinge or CH3 mutations (or mutant forms that lack the residues required for Fab-arm exchange) may decrease IgG4 shuffling and increase the half-life of the IgG4 molecules in vivo (Labrijn, et al, 2009, Nat Biotechnol 27:767-771; Stubenrauch, et al, 2010, Drug Metab Dispos 38:84-91).

The IgG4 variant described herein may therefore contain one or more amino acid substitutions in the IgG4 hinge or CH3 region, wherein IgG4 shuffling is reduced relative to an antibody comprising an IgG4 hinge or CH3 region not comprising said one or more amino acid substitutions. In a specific example, the IgG4 hinge or CH3 region comprises a single amino acid substitution (either at position 228 (e.g. S228P), or at amino acid position 409 (e.g. R409K), or at amino acid position 405 (e.g. F405L) of the heavy chain numbered according to the EU index). In another specific example, the IgG4 hinge or CH3 region comprises two amino acid substitutions (at position 228 (e.g. S228P) and at position 409 (e.g. R409K); or at position 228 (e.g. S228P) and at position 405 (e.g. F405L); or at position 405 (e.g. F405L) and at position 409 (e.g. R409K)) of the heavy chain numbered according to the EU index). In another specific example, the IgG4 hinge or CH3 region comprises three amino acid substitutions (at position 228 (e.g. S228P), at position 405 (e.g. F405L) and at position 409 (e.g. R409K) of the heavy chain numbered according to the EU index). In other specific examples, the IgG4 region additional includes other amino acid substitutions; appropriate substitutions are well known in the art.

Treatment of a Subject

A binding agent (e.g. binding protein such as an antibody) as described herein is useful for treating a condition, disorder and/or symptom which is alleviated by the inhibition of neuromuscular transmission in a subject.

A binding agent (e.g. binding protein such as an antibody) as described herein may also be used to prevent, regulate or reduce skin wrinkling in a subject.

The terms “subject”, “patient” and “individual” are used interchangeably herein.

In certain examples, the subject is a mammal. A subject therefore refers to, for example, dogs, cats, horses, cows, pigs, guinea pigs, mice, rats and the like. The subject may be a primate (e.g. a non-human primate). In a preferred example, the subject is a human.

The subject is typically a subject in need of treatment for a condition, disorder and/or symptom which is alleviated by the inhibition of neuromuscular transmission in a subject.

The binding agent (e.g. binding protein such as an antibody) provided herein may be used for cosmetic (i.e. non-therapeutic) or therapeutic purposes as outlined below.

The binding agent (e.g. binding protein such as an antibody) may advantageously be used to prevent, regulate or reduce skin wrinkling in a subject. In one example, such uses are considered “cosmetic” and/or “non-therapeutic”.

A method of preventing, regulating or reducing skin wrinkling in a subject is therefore provided, wherein the method comprises administering a binding agent (e.g. binding protein such as an antibody) described herein to the subject. In one example, such methods are considered “cosmetic” and/or “non-therapeutic”.

As used herein, the terms “cosmetic” and “non-therapeutic” are used interchangeably and are intended to refer to interventions performed with the intention of addressing (e.g. improving, preventing or regulating) a cosmetic non-pathological condition in a subject. For example, cosmetic treatments may be used to restore or improve the appearance of a subject. Accordingly, a binding agent (e.g. binding protein such as an antibody) described herein may be used for preventing, regulating or reducing skin wrinkling in a subject. Suitable methods for administering a binding agent for this purpose are well known in the art, and include but are not limited to injection.

A method of preventing, regulating or reducing skin wrinkling may also be considered as a method of enhancing relaxation or slackening of cutaneous tissue. The methods may include locally administering to a cutaneous tissue a binding agent (e.g. binding protein such as an antibody) described herein in an amount effective to enhance denervation of the muscle or group of muscles present subcutaneous to the cutaneous tissue to enhance relaxation or slackening of the cutaneous tissue. In some examples, the binding agent (e.g. binding protein such as an antibody) is administered subcutaneously.

Preferably the relaxation or slackening of the cutaneous tissue results in lessening of wrinkles or fine lines of the skin.

Advantageously, binding agents (e.g. binding proteins such as antibodies) described herein may also be used for therapeutic purposes. As used herein, the term “therapeutic” is intended to refer to “treatment” of a subject.

As used herein, the terms “treat”, “treating” and “treatment” are taken to include an intervention performed with the intention of preventing the development or altering the pathology of a condition, disorder or symptom. Accordingly, “treatment” refers to both therapeutic treatment and prophylactic or preventative measures, wherein the object is to prevent or slow down (lessen) the targeted condition, disorder or symptom. As used herein, the terms “disease” and “disorder” are used interchangeably.

As used herein the term “subject” refers to an individual, (e.g., a human or a non-human primate) having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention.

Binding agents (e.g. binding proteins such as antibodies) provided herein may be used for treating or preventing a condition, disorder or symptom which is alleviated by the inhibition of neuromuscular transmission.

As will be clear from the text herein, the binding agents (e.g. binding proteins such as antibodies) described may be used to inhibit neuromuscular transmission. In this context, “inhibition” of neuromuscular transmission encompasses a decrease or reduction in neuromuscular transmission (e.g. when comparing the level of neuromuscular transmission in the absence of the binding agent to the level of neuromuscular transmission in the presence of the binding agent). For the avoidance of doubt, “inhibition”, “inhibit”, “decrease”, “decreased” “reduced” or “reduction” and the equivalent terms are all used herein generally to mean a decrease by a statistically significant amount. “Inhibition”, “inhibit”, “decrease”, “decreased” “reduced” or “reduction” typically means a decrease by at least 10% as compared to a control (i.e. in the absence of binding agent), for example a decrease by at least about 20%, or at least about 30%, or at least about 40%, or at least about 50%, or at least about 60%, or at least about 70%, or at least about 80%, or at least about 90% or up to and including a 100% decrease, or any decrease between 10-100% as compared to a control (i.e. in the absence of binding agent), or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a control (i.e. in the absence of binding agent).

The skilled person will be fully aware of the diseases or conditions which are alleviated by the inhibition of neuromuscular transmission since the use of botulinum toxin A has been in widespread use for medicinal and cosmetic therapies for a number of years (see, for example, Jankovic (2004) Botulinum in clinical practice. J Neurol Neurosurg Psychiatry 75 951-957). In particular, some of the diseases or conditions which are alleviated by the botulinum toxin A mediated inhibition of synaptic transmission are selected from the group consisting of: dystonias, facial spasms, strabismus, muscle hypertonia due to cerebral palsy, stuttering, chronic tension headaches, spasms of the inferior constrictor of the pharynx, pain, migraine, or cosmetic treatments such as reducing wrinkles, brow furrows, etc.

Binding agents (e.g. binding proteins such as antibodies) described herein may be used to treat or prevent a conditions, disorders or symptoms which are alleviated by the inhibition of neuromuscular transmission in a subject. Methods of treating or preventing conditions, disorders or symptoms which are alleviated by the inhibition of neuromuscular transmission are provided, comprising administering a binding agent (e.g. binding protein such as an antibody) described herein to a subject. Accordingly, in vivo methods of treatment are provided, which may be prophylactic and/or therapeutic.

As used herein, treating or preventing a “condition, disorder and/or symptom which is alleviated by the inhibition of neuromuscular transmission” is intended to include treating or preventing, pain secondary to spams or involuntary muscle actions, a neurological disorder or condition in a subject, conditions or diseases resulting from involuntary spasms, muscle spasticity, strabismus, occupational cramps, anal fissures, migraine headaches, brusism, and any combination thereof.

In one example, the pain is pain associated with neuromuscular disorders.

In one example, the condition or disease resulting from involuntary muscle spasms is selected from the group consisting of: hemifacial spasms, blepharospasm, laryngeal dysphoria, head dystonias, neck dystonias, limb dystonias, or rectal spasms.

As used herein, treating or preventing a “condition, disorder and/or symptom which is alleviated by the inhibition of neuromuscular transmission” is intended to also include correcting an external appearance distorted due to excessive muscular activity in a subject.

As used herein, “excessive muscular activity” refers to an increase in muscular activity compared to the norm. Examples of distorted external appearances that may be corrected using a binding agent described herein include muscle spasms and tics.

As used herein, treating or preventing a “condition, disorder and/or symptom which is alleviated by the inhibition of neuromuscular transmission” (also referred to herein as treating or preventing a “condition, disorder or symptom resulting from excessive muscular activity”) is intended to also include treating or preventing a spasm or involuntary contraction in a muscle or a group of muscles in a subject. Non-limiting examples of spasms or involuntary contractions include blepharospasm, strabismus, spasmodic torticollis, focal dystonia, jaw dystonia, occupational dystonia, corneal ulceration (protective ptosis), spasmodic dysphonia (laryngeal dystonia), or facial dyskinesis such as Meige syndrome, hemifacial spasm, aberrant regeneration of facial nerves, or apraxia of eyelid opening.

The inventors have shown that binding agents (e.g. binding proteins such as antibodies) described herein can act as antagonists at the NMJ by inhibiting MuSK phosphorylation. A reduction in MuSK phosphorylation impairs AChR clustering and subsequently blocks neuromuscular transmission. The binding agents (e.g. binding proteins such as antibodies) described herein may therefore be used to induce temporary localised muscle weakness (e.g. paresis) in a subject. Longer term localised muscle weakness/paresis may also be induced (e.g. by multiple rounds of administration). They may therefore be used as an alternative to administration of botulinum neurotoxin.

Botulinum neurotoxin type A (BoNT type A; or BoNT/A) has proven to be of great medical importance due to its ability to cause a very long neuromuscular paresis upon local injections of minute amounts (1 pM concentration) (Pirazzini-M et al., Pharmacol Rev 2017; 69:200). Over the last 30 years, BoNT/A and other botulinum neurotoxins have been successfully exploited for medicinal and cosmetic purposes. These toxins silence NMJs and also can block neurotransmitter release from many types of neurons. Practically every part of the human body including the brain [Botulinum toxin therapy for neuropsychiatric disorders, US 20040180061 A1] can be treated using BoNT/A. An example of a form of BoNT/A that has been used successfully for cosmetic treatment is Botox. Since the paresis of NMJs is reversible, the sustained relaxation of muscles requires repeat injections every three to four months. BoNT/A can block innervation of not only striated muscles but also of smooth muscles. Therefore, botulinum-based treatments have recently expanded to include a dazzling array of nearly a hundred conditions from dystonias to gastrointestinal and urinary disorders.

The binding agents (e.g. binding proteins such as antibodies) described herein may be for administering to a subject in a therapeutically effective amount. “Therapeutically effective amount” means that amount of a drug, compound, antibody, or pharmaceutical binding agent that will elicit the biological or medical response of a subject that is being sought. Accordingly, in therapeutic applications described herein, agents and/or compositions thereof are administered to a subject in an amount sufficient to at least partially arrest the symptoms or disease and its complications. An amount adequate to accomplish this is defined as a “therapeutically effective amount or dose.” Amounts effective for this use will depend on the severity of the disease and the weight and general state of the patient.

A binding agent (e.g. binding protein such as an antibody) described herein may be part of a composition (e.g. a pharmaceutical composition) that comprises the binding agent and one or more other components. A composition may be a composition that comprises a binding agent described herein and a pharmaceutically acceptable excipient, adjuvant, diluent and/or carrier. Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected binding agent without causing any undesirable biological effects or interacting in a deleterious manner with any of the other components of the pharmaceutical composition in which it is contained.

Excipients are natural or synthetic substances formulated alongside an active ingredient (e.g. a binding protein provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

Generally, the binding agents (e.g. binding proteins such as antibodies) are administered in a pharmaceutically effective amount. The amount of binding agent actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions can be administered by a variety of routes including oral, rectal, transdermal, subcutaneous, intravenous, intramuscular, and intranasal. The compositions are typically presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base.

Preferably, the compositions are injectable or transdermal compositions.

The above-described components for pharmaceutical compositions are merely representative. Other materials as well as processing techniques and the like are set forth in Part 8 of Remington's Pharmaceutical Sciences, 17th edition, 1985, Mack Publishing Company, Easton, Pa.

The compounds described herein can also be administered in sustained release forms or from sustained release drug delivery systems. A description of representative sustained release materials can be found in Remington's Pharmaceutical Sciences.

The compounds described herein can be administered as the sole active agent or they can be administered in combination with other agents, including other compounds that demonstrate the same or a similar therapeutic activity and are determined to be safe and efficacious for such combined administration.

Methods

A method of preventing, regulating or reducing skin wrinkling in a subject is provided, the method comprising administering a binding agent (e.g. binding protein such as an antibody) comprising one or more binding regions, wherein only one binding region specifically binds to an Ig-like 1 domain of a MuSK protein to the subject.

The method is considered cosmetic.

Features of appropriate binding agents, subjects, pharmaceutical compositions, means for administration and tests for determining successful administration are described in detail above and apply equally to this aspect.

A method of treating or preventing a condition, disorder and/or symptom which is alleviated by the inhibition of neuromuscular transmission is also provided; the method comprising administering a binding agent (e.g. binding protein such as an antibody) comprising one or more binding regions, wherein only one binding region specifically binds to an Ig-like 1 domain of a MuSK protein, to a subject.

The binding agent is for administration to an appropriate subject in a therapeutically effective amount.

The therapeutically effective amount may be sufficient to decrease MuSK activity in the subject and thereby alleviate a condition, disorder and/or symptom associated with elevated levels (increase or abnormal) of neuromuscular transmission in a subject.

Features of appropriate binding agents, subjects, conditions, disorders and symptoms, pharmaceutical compositions, means for administration and tests for determining successful treatment are described in detail above and apply equally to this aspect.

As used herein, “decreasing” refers to down-regulating.

As used herein, “elevated” refers to higher than normal, increased or up-regulated.

As used herein “MuSK activity” refers to both biological activity and functional activity of a MuSK protein, polypeptide or nucleic acid molecule (e.g., in a MuSK expressing cell or tissue), as determined in vivo, or in vitro, according to standard techniques. MuSK activity includes MuSK dimerization, phosphorylation of MuSK or stimulation of aggregation of AChR on a cell membrane at an NMJ. MuSK activation may result in induction or maintenance of postsynaptic differentiation.

Several methods for measuring neuromuscular transmission are known in the art, for example, electromyographic or electrophysiological evidence may be used.

Preparation of a Medicament

In another aspect, the invention provides for the use of a binding agent (e.g. binding protein such as an antibody) comprising one or more binding regions, wherein only one binding region specifically binds to an Ig-like 1 domain of a MuSK protein in the preparation of a medicament for treating a condition, disorder and/or symptom which is alleviated by the inhibition of neuromuscular transmission in a subject. The medicament is for administration to an appropriate subject in a therapeutically effective amount.

Features of appropriate binding agents, subjects, disorders and symptoms, pharmaceutical compositions, means for administration, and tests for determining successful treatment are described in detail above and apply equally to this aspect.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

The data provided below demonstrates that functionally monovalent MuSK antibodies (isolated from patients) binding the Ig-like 1 domain of MuSK act as antagonists. Conversely, the same antibodies in a bivalent monospecific form act as agonists. As serum IgG4, due to Fab-arm exchange, is also functionally monovalent, and IgG4 patient MuSK antibodies cause MG in animal models and in humans, the monovalency of these antibodies is considered the determining factor in the pathophysiology of MuSK MG.

Results

Description of MuSK MG Patients Included

For the isolation of MuSK-specific B cells peripheral blood mononuclear cells (PBMC) were isolated from two MuSK MG patients. The clinical characteristics of these patients are described in Table 2. The patient that yielded the highest number of MuSK-specific B cells had relapsed after rituximab treatment.

TABLE 2
Clinical characteristics from study subjects at the moment of PBMC withdrawal.

| | | | MuSK | Disease | | |
|---|---|---|---|---|---|---|
| Patient | Age | | titer | severity | Thymus | |
| ID | (y) | Gender | (nmol/L) | (QMG) | status | Treatment |
| 1 | 59 | M | 1.19 | 9/39 | Thymectomy | prednisone, |
| | | | | | 1987 | cellcept, |
| | | | | | No thymoma | pyridostigmine, |
| | | | | | | plasmapheresis, |
| | | | | | | two courses of |
| | | | | | | rituximab |
| | | | | | | 4 and 2 years before |
| 2 | 59 | V | 0.75 | Not | No thymectomy | prednisone, |
| | | | | available | No thymoma | azathioprine, |
| | | | | | | plasmapheresis |

Isolation and genetic characterization of MuSK autoantibodies from MuSK MG patients Antigen-specific single cell sorting yielded eight MuSK-binding B cells from the two MuSK MG patients. The frequency of circulating MuSK clones was ˜7 per 100 million PBMC for patient 1, and 2.5 per 100 million PBMC for patient 2. An overview of the MuSK autoantibody characteristics is given in Table 3. For six out of the eight MuSK autoantibody producing clones the inventors could isolate the variable-region sequences of the heavy chain (V_{H}) and light chain (V_{L}). For one clone only the V_{H} variable-region could be sequenced.

TABLE 3
MuSK autoantibody characteristics

| | Pt | | | VH mutations | | VL mutations | Glycosylation | |
|---|---|---|---|---|---|---|---|---|
| Clone | ID | Isotype | HC VDJ genes | V; J | LC VJ genes | V; J | motif present | Epitope |
| 11-3D9 | 1 | IgG1κ | IGHV3-21*01F | 16; 6 | IGKV1-39*01F | 16; 4 | 1 in CDR1 | Ig-like 1 |
| | | | IGHD3-3*01F | | IGKJ2*01F | | | |
| | | | IGHJ4*02F | | | | | |
| 11-3F6 | 1 | IgG1κ | IGHV3-21*01F | 19; 6 | IGKV1-39*01F | 30; 1 | No | Ig-like 1 |
| | | | IGHD6-19*01F | | IGKJ2*02F | | | |
| | | | IGHJ4*02F | | | | | |
| 11-8G4 | 1 | IgG1κ | IGHV3-21*01F | 34; 5 | N/A | N/A | No | N/A |
| | | | IGHD6-19*01F | | | | | |
| | | | IGHJ4*02F | | | | | |
| 11-7C5 | 1 | | N/A | N/A | N/A | N/A | N/A | N/A |
| 13-3B5 | 1 | IgG4λ | IGHV1-2*02F | 39; 11 | IGLV3-25*03F | 32; 5 | No | Ig-like 1 |
| | | | IGHD3-10*01F | | IGLJ2*01F | | | |
| | | | IGHJ5*02F | | | | | |
| 13-3D10 | 1 | IgG1κ | IGHV3-21*01F | 45; 7 | IGKV1-39*01F | 26; 2 | No | Ig-like 1 |
| | | | IGHD3-16*01F | | IGKJ2*01F | | | |
| | | | IGHJ4*02F | | | | | |
| 13-4D3 | 1 | IgG3κ | IGHV3-21*02F | 31; 6 | IGKV1-39*01F | 12; 3 | No | Ig-like 1 |
| | | | IGHD3-16*01F | | IGKJ2*01F | | | |
| | | | IGHJ4*02F | | | | | |
| 16-8B3 | 2 | IgG1 | N/A | N/A | N/A | N/A | N/A | N/A |

Interestingly, the majority of the antibodies isolated were of the IgG1 isotype. In addition, the inventors identified one IgG4 clone and one IgG3 clone.

Fab-arm exchange is an important feature of IgG4 and might affect the functional characteristics of MuSK autoantibodies (Koneczny et al., 2017). Previous work on polyclonal purified fractions suggested that MuSK MG IgG4 has the ability to undergo Fab-arm exchange (Koneczny et al., 2017). The inventors sequenced the V_{H} of the isolated MuSK monoclonal antibodies and confirmed that these antibodies indeed possess the serine at position 228 and the arginine at position 409 required to undergo Fab-arm exchange (data not shown).

N-linked glycosylation of the Fab can be important for antigen binding, e.g. in rheumatoid arthritis ACPA autoantibodies (van de Bovenkamp et al., 2018). Therefore, the inventors assessed the presence of the NXST (where X can't be a P) motif in the monoclonal MuSK autoantibodies variable regions. In only one clone, such a motif was found, suggesting that glycosylation of the Fab is not essential for all MuSK autoantibodies.

Functional Characteristics of Recombinant MuSK Monoclonal Autoantibodies

Autoantibody binding epitopes are important determinants of the pathomechanism in autoimmune diseases. The Ig-like 1 domain of MuSK was previously recognized as the main immunogenic region of MuSK in polyclonal IgG4 patient antibody fractions and serum (Huijbers et al., 2016). For five of the patient-derived MuSK antibodies the epitope could be mapped to the first Ig-like 1 domain of MuSK (Table 3). No monoclonal autoantibodies against other domains have thus far been identified. To establish the functional characteristics of the MuSK autoantibodies, recombinant antibodies were produced from an IgG1 isolated clone and an IgG4 isolated clone. To assess the importance of autoantibody isotype in MuSK MG each of these variable-regions were cloned in both an IgG1 and IgG4 backbone.

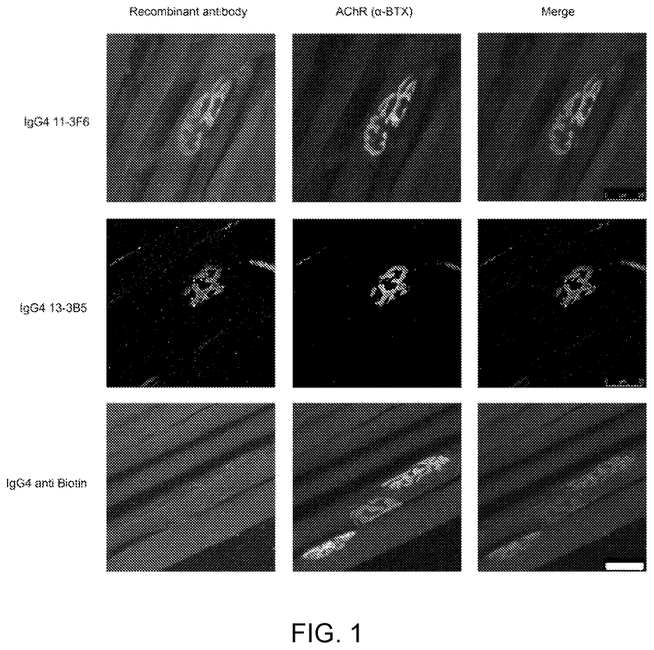

To assess the ability of recombinant patient-derived IgG1 and IgG4 MuSK antibodies to bind to MuSK at the postsynaptic membrane of NMJs, the inventors performed immunostaining on isolated mouse skeletal muscle. Both the IgG1 and IgG4 versions of the recombinant monoclonal antibodies clearly bound to NMJs (FIG. 1, data shown for the IgG4 recombinant antibodies).

Successful neuromuscular transmission depends on properly clustered AChR and is therefore strictly orchestrated through the agrin-Lrp4-MuSK signaling cascade (Burden et al., 2018). Agrin is released by the motor nerve terminal, binds Lrp4 and Lrp4 subsequently binds and stimulates MuSK dimerization and transphosphorylation. Activation of MuSK phosphorylation stimulates a variety of intracellular signaling cascades of which one culminates in AChR clustering. Purified patient IgG4 MuSK autoantibodies inhibit MuSK-Lrp4 interaction, MuSK phosphorylation and thereby reduce AChR clustering in C2C12 myotube cultures and mice (Huijbers & Zhang 2013, Koneczny et al., 2013). Interestingly, when the effect of patient-derived MuSK monoclonal antibodies was assessed on MuSK phosphorylation both recombinant IgG1 and IgG4 (bivalent monospecific) antibodies strongly activated rather than inhibited MuSK phosphorylation (FIG. 2A). This effect was observed in both absence and presence of agrin. Activation of MuSK phosphorylation was furthermore concentration-dependent (FIG. 2B) and differed slightly between the two clones. Monovalent Fab-antibody fragments produced by papain digestion from the same antibodies inhibited MuSK phosphorylation (FIG. 2C).

Recombinant monoclonal IgG1 and IgG4 however both engage in bivalent monospecific antibody-antigen interactions. To investigate the functional effects of the bispecificity and functional monovalency of Fab-arm exchanged IgG4 MuSK antibodies in patients, the inventors generated monovalent Fab fragments from these recombinant antibodies by papain digestion. In vitro, these Fab fragments inhibited agrin-dependent MuSK phosphorylation (FIG. 2C) and AChR clustering similar to patient serum-derived anti-MuSK IgG4 (FIG. 2D).

In contrast, (and in line with activating MuSK phosphorylation in vitro), bivalent monospecific monoclonal MuSK antibodies activated agrin-dependent AChR clustering compared to Fab fragments. Furthermore, AChR clustering could be partially induced using bivalent monospecific antibodies independent from agrin (FIG. 2D). Thus, monovalent MuSK binding blocks the AChR clustering pathway, whereas bivalent monospecific MuSK antibodies stimulate MuSK, and can facilitate or induce AChR clustering in this tissue culture model.

These observations suggest that an agent with two MuSK Ig-like 1 domain binding sites has the ability to activate MuSK, whereas agents with only one MuSK Ig-like 1 domain binding site (similar to what was seen with patient purified IgG4 or Fab fragments) inhibit MuSK activation.

MuSK Antibody Characterisation

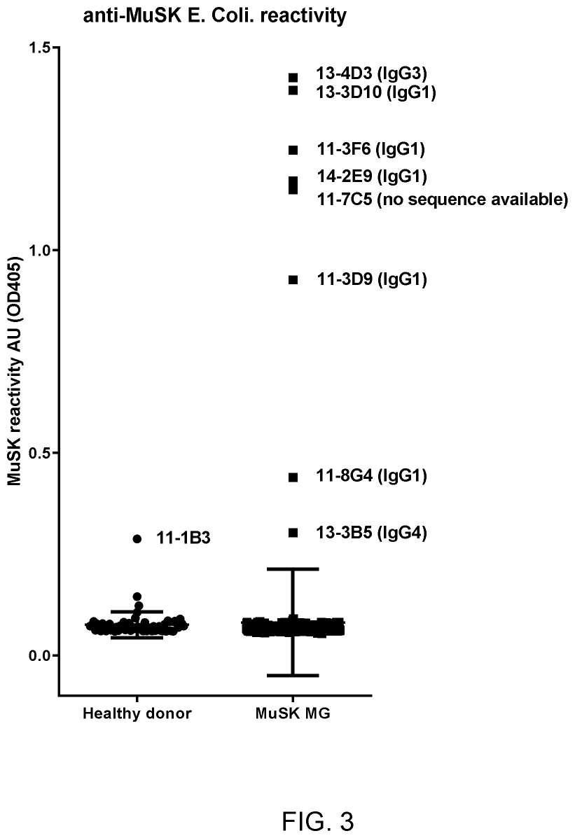

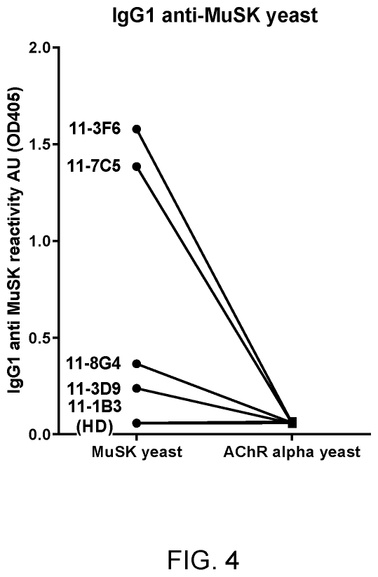

MuSK antibodies (isolated from myasthenia gravis patients with MuSK serum antibodies) that bind to the Ig-like 1 domain of MuSK have been isolated and sequenced as detailed below. As shown in FIGS. 3 and 4, a total of eight MuSK Ig-like 1 domain reactive antibodies (11-309, 11-3F6, 11-8G4, 13-3B5, 13-3D10, 13-403, 14-2E9 and 11-705) were obtained from MuSK MG patients.

Variable Region Sequences/Binding Regions Specific for Binding to an Ig-Like 1 Domain of a MuSK Protein

The variable region amino acid sequence for seven of these clones was obtained (11-3D9, 11-3F6, 11-8G4, 13-3B5, 13-3010, 13-4D3 and 14-2E9). The variable region of an antibody is typically made up of the heavy chain variable domain (VH) and the light chain variable domain (VL). Each of VH and VL typically have a CDR1, a CDR2 and a CDR3 sequence that, together, confer specificity for the target antigen (i.e. VH CDR1, VH CDR2, VH CDR3, VL CDR1, VL CDR2 and VL CDR3 together confer specificity for the target antigen). However, as stated previously herein, a single VH or VL domain (i.e. VH CDR1, VH CDR2 and VH CDR3; or VL CDR1, VL CDR2 and VL CDR3) may also be sufficient to confer antigen-binding specificity.

For six clones, both the heavy chain and light chain variable domains were successfully sequenced—for one clone (11-8G4) only the heavy chain variable domain sequence was successfully obtained. The heavy chain and light chain variable domain sequences are provided for each clone below. The type of light chain is also indicated below. HC=Heavy chain; LC=Light chain; VH=heavy chain variable domain; VL=light chain variable domain.

Amino acid sequences that are underlined represent CDRs (from N to C terminus of the amino acid sequence: CDR1, CDR2, CDR3).

The antibody sequences have been analysed using IMGT/V-QUEST program version: 3.4.17 (19 Feb. 2019)—IMGT/V-QUEST reference directory release: 201910-2 (5 Mar. 2019) (http://imgt.org/IMGT_vquest/vquest) Selecting for Homo sapiens sequences.

11-3D9 VH
(SEQ ID NO: 9)
EVQLVESGGGLVKPGESLRLSCAASGFNFSTYTMHWVRQAPGKGLEWVSS
ISSRSAYKYYADSVKGRFTISRDIAKNSLYLQMNSLRAEDTAVYYCARDF
FQLGPPRFDSWGQGTLVTVSS

In other words, the heavy chain variable domain (VH) CDRs may be as follows: CDR1 comprising GFNFSTYT (SEQ ID NO:10), CDR2 comprising ISSRSAYK (SEQ ID NO: 11) and CDR3 comprising ARDFFQLGPPRFDS (SEQ ID NO:12). These CDRs may optionally be in the context of a VH comprising the sequence of SEQ ID NO:9.

11-3D9 VLk (SEQ ID NO: 13):
DIQMTQSPSSLSASIGDRVTISCRASQRISSFLNWYQQKPGKPPNLLIYG
ASNLQSGVPSRFSGSGSGTDFTLTITSLQPEDYAIYYCQQSYSPMYTFGQ
GTSLEIK

In other words, the light chain variable domain (VL) CDRs may be as follows: CDR1 comprising QRISSF (SEQ ID NO:14), CDR2 comprising GAS (SEQ ID NO: 15) and CDR3 comprising QQSYSPMYT (SEQ ID NO:16). These CDRs may optionally be in the context of a VL comprising the sequence of SEQ ID NO:13.

Antigen specificity may be obtained by a combination of CDRs of SEQ ID NO: 10, 11 and 12 (heavy chain) and CDRs of SEQ ID NO: 14, 15 and 16 (light chain). For example, antigen specificity may be obtained by a combination of variable domains of SEQ ID NO: 9 (heavy chain) and SEQ: 13 (light chain).

11-3F6 VH (SEQ ID NO: 17):
EVQLVESGGGLVKPGGSLRLSCAASGFTFSSYTMDWVRQAPGKGLEWVSS
IGSNGDYIYYADSVRGRFTISRDNAKNSLYLQMNSLRPDDTADYYCARGQ
LAVAGTHFDYWGRGSLVTVSS

In other words, the heavy chain variable domain CDRs may be as follows: CDR1 comprising GFTFSSYT (SEQ ID NO:18), CDR2 comprising IGSNGDYI (SEQ ID NO: 19) and CDR3 comprising ARGQLAVAGTHFDY (SEQ ID NO:20). These CDRs may optionally be in the context of a VH comprising the sequence of SEQ ID NO:17.

11-3F6 VLk (SEQ ID NO: 21):
DIQMTQSPSSLSASVGDRVTISCRASQKVNKYVNWYQQTPGKAPRLLIYA
ASTLQSGVPSRFSGSGSGA [symbol] LTISGLQPEDFAIYFCQQSYSPLCTFGQ
GTKLEIK

In other words, the light chain variable domain CDRs may be as follows: CDR1 comprising QKVNKY (SEQ ID NO:22), CDR2 comprising AAS (SEQ ID NO: 23) and CDR3 comprising QQSYSPLCT (SEQ ID NO:24). These CDRs may optionally be in the context of a VL comprising the sequence of SEQ ID NO:21.

Antigen specificity may be obtained by a combination of CDRs of SEQ ID NO: 18, 19 and 20 (heavy chain) and CDRs of SEQ ID NO: NO: 22, 23 and 24 (light chain). For example, antigen specificity may be obtained by a combination of variable domains of SEQ ID NO:17 (heavy chain) and SEQ: 21 (light chain).

11-8G4 VH
(No VL sequence available for this antibody)
(SEQ ID NO: 25)
EVQLVESGGGLVKPGGSLRLSCAASGFTFSDFTMNWVRQAPGQGLEWVSS
IGSSGTFIYYAASVRGRFTISRDNAQDLLSLQMNSLRADDTATYFCARGR
IAVAGTHFDLWGQGTLVTVSS

In other words, the heavy chain variable domain CDRs may be as follows: CDR1 comprising GFTFSDFT (SEQ ID NO:26), CDR2 comprising IGSSGTFI (SEQ ID NO: 27) and CDR3 comprising ARGRIAVAGTHFDL (SEQ ID NO:28). These CDRs may optionally be in the context of a VH comprising the sequence of SEQ ID NO:25.

13-3B5 VH (SEQ ID NO: 29):
QVQLVQSGAVVAKPGASVQVSCKTSGYTFTGQYLHWVRQAPGQGLEWIGW
INPSSGVTKFAEKFEGRATMTRDTSITTAYIDLRSLRSDDTATYYCATLS
LGVYYVGMVAWGQGTLVTVSS

In other words, the heavy chain variable domain CDRs may be as follows: CDR1 comprising GYTFTGQY (SEQ ID NO:30), CDR2 comprising INPSSGVT (SEQ ID NO: 31) and CDR3 comprising ATLSLGVYYVGMVA (SEQ ID NO:32). These CDRs may optionally be in the context of a VH comprising the sequence of SEQ ID NO:29.

13-3B5 VLI variable region
(SEQ ID NO: 33)
SYELTQPPSVSVSPGQTATITCSGDGLAQQHVYWFQQRPGQAPLLIIYKD
IERPSGIPERFSGSSSGTTAMLTISGVQAEDEADYYCQSGDRTATSVLFG
GGTKMTVL

In other words, the light chain variable domain CDRs may be as follows: CDR1 comprising GLAQQH (SEQ ID NO:34), CDR2 comprising KDI (SEQ ID NO: 35) and CDR3 comprising QSGDRTATSVL (SEQ ID NO:36). These CDRs may optionally be in the context of a VL comprising the sequence of SEQ ID NO:33.

Antigen specificity may be obtained by a combination of CDRs of SEQ ID NO: 30, 31 and 32 (heavy chain) and CDRs of SEQ ID NO: 34, 35 and 36 (light chain). For example, antigen specificity may be obtained by a combination of variable domains of SEQ ID NO: 29 (heavy chain) and SEQ: 33 (light chain).

13-3D10 VH
(SEQ ID NO: 37)
EDLLVESGGGLVKPGQSLTLSCAASGFDFSASTMAWVRQAPGKGLEWVAS
VSGDSHHIYYADSLKGRFTLSRDNARNSFFLEMNSLRAEDTAVYYCARER
LLRLGVGFDSWGQGSLVAVSS

In other words, the heavy chain variable domain CDRs may be as follows: CDR1 comprising GFDFSAST (SEQ ID NO:38), CDR2 comprising VSGDSHHI (SEQ ID NO: 39) and CDR3 comprising ARERLLRLGVGFDS (SEQ ID NO:40). These CDRs may optionally be in the context of a VH comprising the sequence of SEQ ID NO:37.

13-3D10 VLk
(SEQ ID NO: 41)
DIHMTQSPSSLSSSVGDRVTMTCRASQRISGFVNWYQQKPGRAPTLLISA
ASTLQSGVPSRFSGSASGTDFTLTISGLQPEDSAIYYCQQSYSPLYTFGQ
GTKVEIK

In other words, the light chain variable domain CDRs may be as follows: CDR1 comprising QRISGF (SEQ ID NO:42), CDR2 comprising AAS (SEQ ID NO: 43) and CDR3 comprising QQSYSPLYT (SEQ ID NO:44). These CDRs may optionally be in the context of a VL comprising the sequence of SEQ ID NO:41.

Antigen specificity may be obtained by a combination of CDRs of SEQ ID NO: 38, 39 and 40 (heavy chain) and CDRs of SEQ ID NO: 42, 43 and 44 (light chain). For example, antigen specificity may be obtained by a combination of variable domains of SEQ ID NO: 37 (heavy chain) and SEQ: 41 (light chain).

13-4D3 VH
(SEQ ID NO: 45)
EVQLMESGGGLVKPGGSLRLSCTASGFTFSSYTMTWVRQAPGKGLEWVSS
ISSGGHYIYYTDSLKGRFIISRDNAKNSVFLQMNNLRAEDTATYYCARER
LLRLGVGFDFWGQGSLVTVSS

In other words, the heavy chain variable domain CDRs may be as follows: CDR1 comprising GFTFSSYT (SEQ ID NO:46), CDR2 comprising ISSGGHYI (SEQ ID NO: 47) and CDR3 comprising ARERLLRLGVGFDF (SEQ ID NO:48). These CDRs may optionally be in the context of a VH comprising the sequence of SEQ ID NO:45.

13-3D4 VLk
(SEQ ID NO: 49)
DIQMTQSPSSLSASEGDRVTMTCRASQSISGYLNWYQQKPGKAPKLLIYA
ASTLQSGVPSRFSGSSSGTEFTLSISSLQPEDFATYYCQQSYSALYTFGQ
GTRVEIK

In other words, the light chain variable domain CDRs may be as follows: CDR1 comprising QSISGY (SEQ ID NO:50), CDR2 comprising AAS (SEQ ID NO: 51) and CDR3 comprising QQSYSALYT (SEQ ID NO:52). These CDRs may optionally be in the context of a VL comprising the sequence of SEQ ID NO:49.

Antigen specificity may be obtained by a combination of CDRs of SEQ ID NO: 46, 47 and 48 (heavy chain) and CDRs of SEQ ID NO: 50, 51 and 52 (light chain). For example, antigen specificity may be obtained by a combination of variable domains of SEQ ID NO: 45 (heavy chain) and SEQ: 49 (light chain).

14-2E9 VH
(SEQ ID NO: 53)
EVQLVESGGGLVQPGGSLRLSCAASGFTFSSYWMHWVRQVPGKGLVWVSR
LNEDGSTTNYADSVKGRFTISRDNAKYTLYLQMNSLRFEDTAVYYCVSDL
SGKDEHWGQGTLVTVSS

In other words, the heavy chain variable domain CDRs may be as follows: CDR1 comprising GFTFSSYW (SEQ ID NO:54), CDR2 comprising LNEDGSTT (SEQ ID NO: 55) and CDR3 comprising VSDLSGKDEH (SEQ ID NO:56). These CDRs may optionally be in the context of a VH comprising the sequence of SEQ ID NO:53.

14-2E9 LCk
(SEQ ID NO: 57)
DIVMTQSPLSLPVTPGEPASISCRSTQSLLHSNGYYWLDWYLQKPGQSPQ
LLVYLGFNRASGVPDRFSGSGSGTDFTLKISRVEAEDVGVYYCMQGLQTP
YTFGQGTTLEIK

In other words, the light chain variable domain CDRs may be as follows: CDR1 comprising QSLLHSNGYYW (SEQ ID NO:58), CDR2 comprising LGF (SEQ ID NO: 59) and CDR3 comprising MQGLQTPYT (SEQ ID NO:60). These CDRs may optionally be in the context of a VL comprising the sequence of SEQ ID NO:57.

CDRs of SEQ ID NO: 54, 55 and 56 (heavy chain) may be together with CDRs of SEQ ID NO: 58, 59 and 60 (light chain). Variable domains of SEQ ID NO: 53 (heavy chain) and SEQ: 57 (light chain) may also be combined.

Antigen specificity may be obtained by a combination of CDRs of SEQ ID NO: 54, 55 and 56 (heavy chain) and CDRs of SEQ ID NO: 58, 59 and 60 (light chain). For example, antigen specificity may be obtained by a combination of variable domains of SEQ ID NO: 53 (heavy chain) and SEQ: 57 (light chain).

Fc Region Sequences

From 5 clones sufficient cDNA was left to determine the Fc sequence of the antibody. See below for the subclass analysis. The reference sequences were obtained from ensemble.org.

Reference sequences
IgG1
(SEQ ID NO: 61)

[symbol]

[symbol]

[symbol]

[symbol]

[symbol]

GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPELQLEESCAEAQDGELDGLWTTITIFITLFLL
SVCYSATVTFFKVKWIFSSVVDLKQTIIPDYRNMIGQGA
IgG2
(SEQ ID NO: 62)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDISVEWESNGQPENNYKTTPPMLDSDGSFFLYSKLTVDKSRWQQGN
VFSCSVMHEALHNHYTQKSLSLSPGK
IgG3
(SEQ ID NO: 63)
XSTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
IgG4
(SEQ ID NO: 64)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK

cDNA generated from the B cell clones was sent for sanger sequencing. This gave clear data with regard to the antibody Fc sequence. The DNA sequence was translated using https://web.expasy.org/translate/.

11-3D9 clone amino acid sequence
(translated from cDNA)
SEQ ID NO: 65
GDKRVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TVTR
11-8G4 clone amino acid sequence
(translated from cDNA)
SEQ ID NO: 66
GDKRVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKL
TV
13-3D10 clone amino acid sequence
(translated from cDNA)
SEQ ID NO: 67
KRVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTV
T
13-4D3 clone amino acid sequence
(translated from cDNA)
SEQ ID NO: 68
RVELKTPLGDTTHTCPRCPEPKSCDTPPPCPRCPEPKSCDTPPPCPRCPE
PKSCDTPPPCPRCPAPELLGGPSVFLFPPKPKDTLMISRTPEVTCVVVDV
SHEDPEVQFKWYVDGVEVHNAKTKPREEQFNSTFRVVSVLTVLHQDWLNG
KEYKCKVSNKALPAPIEKTISKTKGQPREPQVYTLPPSREEMTKNQVSLT
CLVKGFYPSDIAVEWESSGQPENNYNTTPPMLDSDGSFFLYSKLTV
13-3E35 clone amino acid sequence
(translated from cDNA)
SEQ ID NO: 69
DKRVESKYGPPCPSCPAPEFLGGPSVFLFPPKPKDTLMISRTPEVTCVVV
DVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVTR

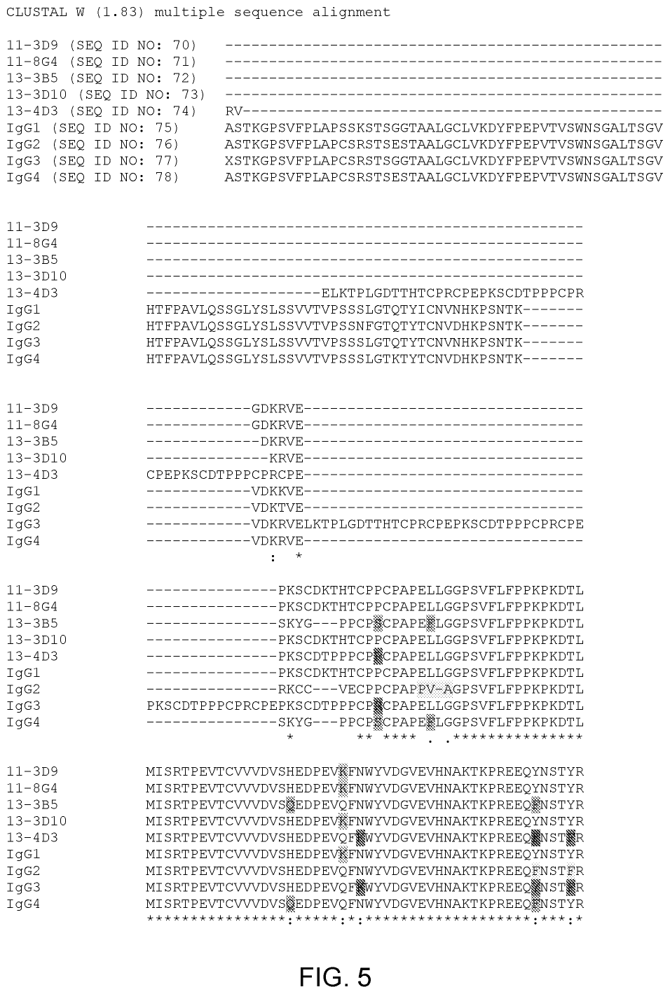

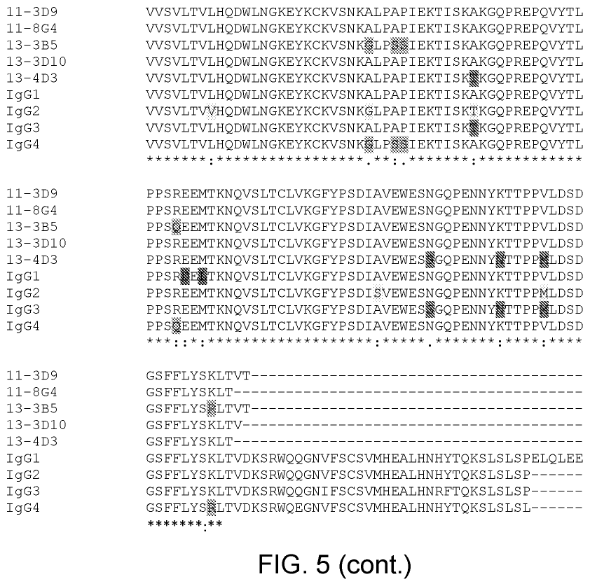

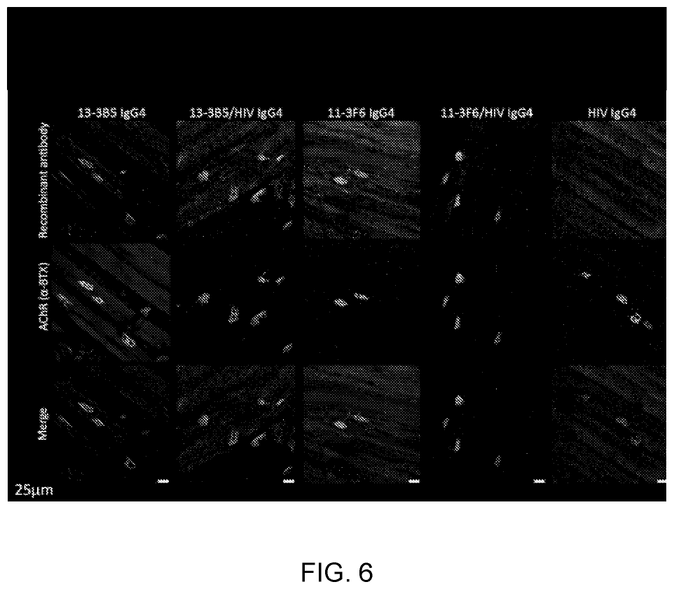

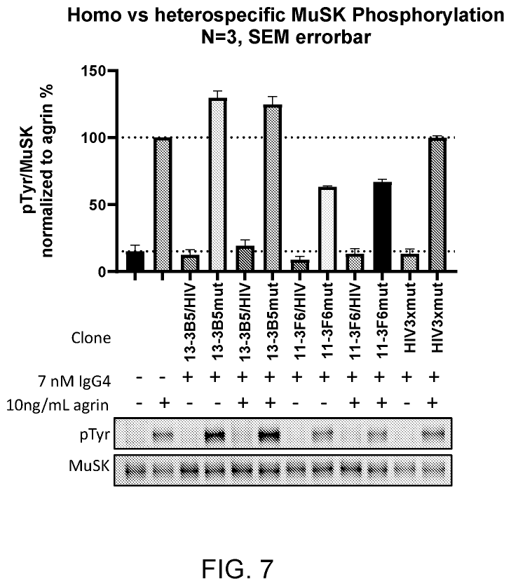

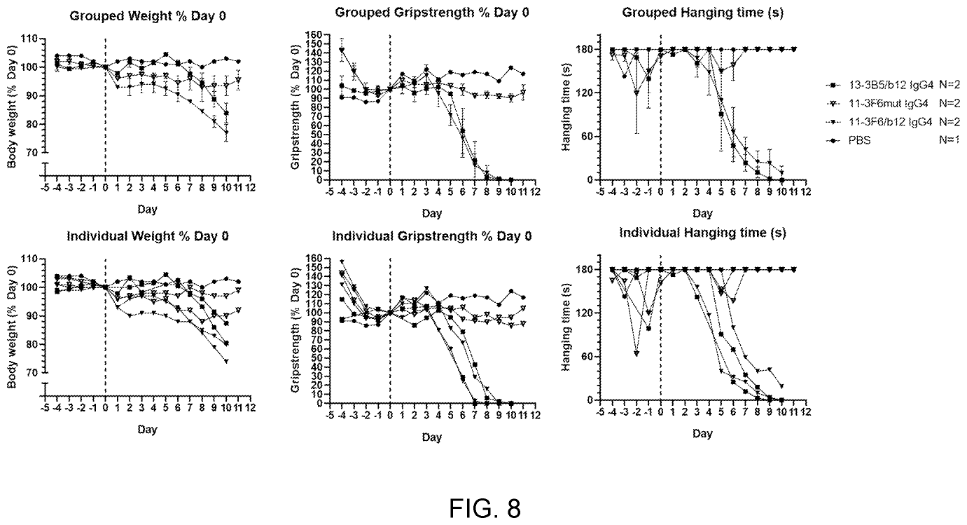

Using the multiple sequence alignment program Clustal W (1.83), the inventors conclude that 11-3D9, 11-8G4, 13-3D10 all have a standard IgG1 sequence (See FIG. 5). 13-3B5 has a standard IgG4 Fc tail and 13-4D3 has a IgG3 Fc sequence. All variants for 13-4D3 are known on ensemble.org. The colour coding is to indicate similarities between different antibodies and their matching subclass reference sequence. The subclass specificity of 11-3F6 and 14-2E9 was also determined in ELISA with IgG1 specific antibody (see FIG. 3).

Discussion

One of the most striking and surprising findings of this study is the fact that (patient-derived) MuSK antibody valency and consequently MuSK autoantibody isotype are key determinants in their ability to inhibit MuSK function and induce MG. Bivalent monospecific MuSK recombinant antibodies binding the Ig-like 1 domain of MuSK acted as agonists and activate MuSK phosphorylation (and AChR clustering) in myotube cultures independent from agrin. Both original IgG1 and IgG4 antibodies showed this effect. In contrast, functionally monovalent Fab fragments generated from these antibodies inhibit agrin-dependent MuSK phosphorylation (and AChR clustering) similar to purified patient IgG4 and IgGtotal (Huijbers & Zhang et al. 2013, Koneczny et al., 2013). The ability of patient IgG4 to undergo Fab-arm exchange thus seems crucial for the pathogenesis of disease as Fab-arm exchange renders endogenously produced MuSK IgG4 bi-specific and functionally monovalent for the MuSK antigen. Previous research has suggested patient IgG4 has the ability to undergo Fab-arm exchange (Koneczny et al. 2017 J autoimmunity) and the sequences of the MuSK monoclonals described herein confirm that the required residues for this process are present. The data suggests that bivalent monospecific MuSK antibodies binding the MuSK Ig-like 1 domain have the ability to dimerize and activate MuSK, whereas functionally monovalent MuSK Ig-like 1 domain antibodies have the ability to inhibit MuSK dimerization and thus inhibit MuSK activation.

To summarize, MuSK antibody valency is a key determinant for the functional effects. Bivalent monospecific antibodies, with two MuSK Ig-like 1 domain binding sites, stimulate MuSK, while monovalent or bi-specific antibodies, with only one MuSK Ig-like 1 domain binding site, block MuSK function.

In conclusion, the results suggest that functional monovalency of MuSK antibodies is crucial for the induction of myasthenic features.

Material & Methods

Patient Selection

MuSK MG patients were recruited in our MG outpatient clinic at the Leiden University Medical Center and were selected based on the presence of a positive MuSK antibody test (RSR ltd) and clinical characteristics consistent with MG. The study was conducted in accordance with the Declaration of Helsinki, was approved by the local medical ethics committee and all patients signed informed consent.

Isolation of Monoclonal Autoantibodies from MuSK MG Patients

MuSK-binding memory B cells were isolated from cryopreserved PBMC selecting for CD19⁺, CD20⁺, CD27⁺ cells (CD19-BV421 H1B19 BD, CD20-AF700 2H7 BD, CD27-APCHy7 M-T271 BD, 0.1% BSA, 2 mM EDTA/dPBS, all monoclonals were mouse anti human). To remove dead cells and non-B cells a dump channel was included (7-AAD,00-6993-50, CD3/FITC UCHT1 BD and CD14/FITC M5E2, BD and CD56/FITC HCD56 Biolegend). Selection for antigen specific cells was done by using recombinant MuSK produced in E. Coli (Huijbers et al., 2016) labeled with R-PE (AS-72113, Anaspec) and MuSK produced in yeast tagged with DyLight 650 (a kind gift of Kostas Lazardinis and Socrates Tzartos, Dylight650 NHS ester, Thermofisher). Single cells were cultured on irradiated L-CD40L feeder cells (a kind gift from Kees van Kooten) in a 96 wells plate in RPMI medium supplemented with 1% pencilline, streptomycin,—50 uM beta-mercaptoethanol (M3148, Sigma), 20 ug/ml human apo-transferrin, (T2036, Sigma-Aldrich, depleted for human IgG with protein A sepharose [GE Healthcare (1 ng/ml IL1b (201-LB-005, R&D), 50 ng/ml IL-21 (PHC0215, Thermofisher), 0.3 ng/ml TNFa (210-TA-005, Thermofisher), 0.5 ug/ml R848 (SML0196, Sigma) (Lighaam et al., 2014). On average 50 million PBMC were used per patients to isolate between 1-6 antigen-specific cells. After two weeks, the medium was tested for MuSK reactive antibodies using the MuSK ELISA described previously (Huijbers et al., 2016). In short, recombinant MuSK was coated overnight on maxisorp plates (NUNC). The next day the plates were washed with PBS and blocked with 2% casein 0.01% tween in PBS. To detect MuSK-binding antibodies culture medium was incubated 1:10 in block buffer and bound antibodies were detected using alkaline phosphatase labelled goat anti-human antibody (Rabbit anti human IgG AP, 309-055-008 Jackson Laboratories) and PNP as a substrate. Plates were read on a Biotek plate reader at 405 nm.

RNA Isolation, cDNA Production and Antibody Sequence Isolation

After two weeks, plates were spun and supernatant was removed to perform the antibody analysis described above. Single wells containing MuSK antibody producing cells were lysed with 150 μl Qiazol and RNA was isolated using standard chloroform extraction and isopropanol/ethanol precipitation. RNA was rehydrated in 8 μl H₂O and stored at −80° C. until further use. cDNA was directly synthesized (without pre-amplification or purification) using Smartscribe reverse transcriptase (Takara Bio Europe) an OligodT40 primer and the template switching oligo for 10 rounds of amplification (primer sequences in table 4). Full length V(D)J were obtained by ARTISAN PCR. Koning et al., 2017. HC primer stands for heavy chain.

TABLE 4
Primer sequences.

| | | SEQ ID |
|---|---|---|
| Primer | Sequence | NO: |
| cDNA synthesis | | |
| Oligo- | 5′-AAGCAGTGGTATCAACGCAGAGTACT | 3 |
| dT40VN | 40VN-3′ | |
| BC-TSO | 5′-AAGCAGTGGTATCAACGCAGAGTACA | 4 |
| | TrGrG+G-3′ | |
| Gene specific amplification | | |
| General | 5′-CTTAAGCAGTGGTATCAACGCAGAGT | 5 |
| Fw | ACATG-3′ | |
| IgG HC | 5′-GGAAGGTGTGCACGCCGCTGGTC-3′ | 6 |
| rev | | |
| hkappaLC | 5′-CTGATGGGTGACTTCGCMG-3′ | 7 |
| rev | | |
| hlambdaLC | 5′-CACACYAGTGTGGCCTTGTTGGCTT | 8 |
| rev | G-3′ | |

Where “dT40” stands for 40 T nucleotides followed by VN where “V” stands for either an A, C or G nucleotide and “N” stands for any nucleotide (see https://Www.mathworks.com/help/bioinfo/ref/baselookup.html). In the BC-TSO “rG” stands for riboguanosines and the G+ stands for one LNA-modified guanosine. In the hkappaLC rev primer “M” stands for an A or C nucleotide and in the hlambdaLC rev primer “Y” stands for C or T nucleotide.

Recombinant Antibody Production, Purification and Characterization

Heavy and light chain sequences were ordered at Geneart (Thermofisher) in an IgG1 and IgG4 backbone pcDNA3.1 vector and transfected in suspension Freestyle HEK293-F cells (R790-07, Thermofisher) using Fectin (12347-019, Thermofisher) in FreeStyle™ 293 Expression Medium (12338, ThermoFisher). To increase transfection and production efficiency the cells were co-transfected with SV40 large T antigen, hp21 and hp27 (A kind gift from Theo Rispens). After 6 days culture medium was collected, cell debris was removed by centrifugation and IgG was purified using a HiTrap™ Protein A affinity column (17-0402-01, GE healthcare) on an Akta pure (GE Healthcare). Antibodies were dialyzed to PBS, filter-sterilized and stored at −20° C. Fab fragments were generated from these recombinant antibodies using papain according to manufacturer's instructions (20341, ThermoFisher) with the following adjustments: Input concentration of the antibodies was 0.5 mg/mL and the duration of the digest was 1 hour for 13-3B5 and 3 hours for 11-3F6 and anti-biotin (13-3B5 was lost when digest took longer than 1 hour). Total IgG and Fc fragment depletion was performed for 2 hours head over head at RT with protein A agarose beads (11134515001, Roche).

To determine that equal amounts of MuSK-specific Fabs were used, concentration was measured compared to their parent antibody in the MuSK ELISA replacing the above described antibody combinations with a mouse anti-human Fab (1:1000, SA1-19255, Pierce) and a rabbit anti-mouse alkaline phosphatase antibody (1:750, D0314, Dako). Biotin Fab concentration was determined comparing MuSK Fab concentration on western blot using the same primary antibody and a donkey anti-mouse 800CW (1:10.000, 925-32212, Licor). Bound antibodies were detected using the Odyssey CLx (Licor).

To determine the binding characteristics of the recombinant and medium produced antibodies we performed an epitope mapping ELISA as described previously (Huijbers et al., 2016). Truncated versions of the MuSK proteins were immobilized on maxisorp plates and exposed to either antibodies from the single cell cultures 1:10 or to recombinant antibodies. Recombinant antibodies were also used to immunostain mouse levator auris longus muscle overnight at 4° C. at 1:100. AChRs in synaptic regions were labelled with AlexaFluor488 conjugated alpha-bungarotoxin (Life technologies) and bound recombinant antibodies were detected with AlexaFluor594 conjugated goat anti-human IgG (Life technologies). Muscles were imaged on a Leica SP8 confocal laser-scanning microscope and analyzed using Las X software.

To determine the ability of the antibodies to inhibit MuSK phosphorylation and AChR clustering we exposed C2C12 myotubes cultures (Cell lines service) to them as described previously (Huijbers & Zhang et al 2013 PNAS). Differentiated myotubes were stimulated with 0.1 nM agrin (550-AG-100, R&D systems) in the presence or absence of 100 ng/mL recombinant antibodies or Fab fragments. For MuSK phosphorylation data, immunoprecipitation of MuSK was initiated after 30 minutes of exposure. Then the myotubes were lysed and MuSK was precipitated using 5 μL/sample rabbit anti MuSK polyclonal serum (ab94276, or ab94277 a kind gift of Steve Burden) during an overnight incubation at 4° C. (Huijbers & Zhang et al. 2013 PNAS). Bound antigen-antibody complexes were precipitated using protein A agarose beads (11134515001, Roche) which were extensively washed. Samples were subsequently ran on SDS-PAGE gel and transferred to PDVF membrane. MuSK and phosphorylated MuSK was detected using goat anti-rat MuSK (1:2000, AF562, R&D systems) and mouse anti-phosphotyrosine clone 4G10 (1:1000, 05-321, Millipore) as primary antibodies, and donkey anti-mouse-680RD (1:10,000, 926-68072, Licor) and donkey anti-goat 800CW (1:10,000, 926-32214, Licor). Bound antibodies were detected using the Odyssey CLx (Licor).

AChR clustering was studied after 16 hours of exposing myotubes to 100 ng/mL recombinant antibodies or Fab fragments in absence or presence of 0.1 nM agrin. Subsequently the cells were washed three times with differentiation medium (DMEM, 31966 Gibco, 2% heat-inactivated horse serum 26050-088, Gibco, 1% pen/strep and 1% L-glutamine) and incubated with 0.5 μg/mL AlexaFluor488 conjugated α-bungarotoxin (B13422, ThermoFisher) in differentiation medium for 30 minutes at 37° C. After staining cells were fixed in 4% formalin solution for 5 minutes, washed with PBS and mounted using hardset mounting medium (H-1500, Vector laboratories). Twenty fields divided over two coverslips per condition were randomly selected, and imaged with Leica DM5500 microscope. AChR cluster count and size were analyzed using ImageJ (1.48v).

Statistics

All data are expressed as mean±standard error of the mean (SEM). Statistical significance of differences between treatment groups were tested with Student's t-tests, with corrections for multiple testing wherever appropriate. Differences with P-values <0.05 were considered statistically significant.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Sequences:
MuSK (human - isoform 205):
SEQ ID NO: 1
MRELVNIPLVHILTLVAFSGTEKLPKAPVITTPLETVDALVEEVATFMCA
VESYPQPEISWTRNKILIKLFDTRYSIRENGQLLTILSVEDSDDGIYCCT
ANNGVGGAVESCGALQVKMKPKITRPPINVKIIEGLKAVLPCTTMGNPKP
SVSWIKGDSPLRENSRIAVLESGSLRIHNVQKEDAGQYRCVAKNSLGTAY
SKVVKLEVEVFARILRAPESHNVTFGSFVTLHCTATGIPVPTITWIENGN
AVSSGSIQESVKDRVIDSRLQLFITKPGLYTCIATNKHGEKFSTAKAAAT
ISIAEWSKPQKDNKGYCAQYRGEVCNAVLAKDALVFLNTSYADPEEAQEL
LVHTAWNELKVVSPVCRPAAEALLCNHIFQECSPGVVPTPIPICREYCLA
VKELFCAKEWLVMEEKTHRGLYRSEMHLLSVPECSKLPSMHWDPTACARL
PHLDYNKENLKTFPPMTSSKPSVDIPNLPSSSSSSFSVSPTYSMTVIISI
MSSFAIFVLLTITTLYCCRRRKQWKNKKRESAAVTLTTLPSELLLDRLHP
NPMYQRMPLLLNPKLLSLEYPRNNIEYVRDIGEGAFGRVFQARAPGLLPY
EPFTMVAVKMLKEEASADMQADFQREAALMAEFDNPNIVKLLGVCAVGKP
MCLLFEYMAYGDLNEFLRSMSPHTVCSLSHSDLSMRAQVSSPGPPPLSCA
EQLCIARQVAAGMAYLSERKFVHRDLATRNCLVGENMVVKIADFGLSRNI
YSADYYKANENDAIPIRWMPPESIFYNRYTTESDVWAYGVVLWEIFSYGL
QPYYGMAHEEVIYYVRDGNILSCPENCPVELYNLMRLCWSKLPADRPSFT
SIHRILERMCERAEGTVSV
The Ig-like 1 domain of the human
MuSK protein:
SEQ ID NO: 2
PVITTPLETVDALVEEVATFMCAVESYPQPEISWTRNKILIKLFDTRYSI
RENGQLLTILSVEDSDDGIYCCTANNGVGGAVESCGALQV

| | | SEQ ID |
|---|---|---|
| Primer | Sequence | NO: |
| cDNA synthesis | | |
| Oligo- | 5′-AAGCAGTGGTATCAACGCAGAGTACT40V | 3 |
| dT40VN | N-3′ | |
| BC-TSO | 5′-AAGCAGTGGTATCAACGCAGAGTACATrG | 4 |
| | rG+G-3′ | |
| Gene specific amplification | | |
| General | 5′-CTTAAGCAGTGGTATCAACGCAGAGTACA | 5 |
| Fw | TG-3′ | |
| IgG HC | 5′-GGAAGGTGTGCACGCCGCTGGTC-3′ | 6 |
| rev | | |
| hkappaLC | 5′-CTGATGGGTGACTTCGCMG-3′ | 7 |
| rev | | |
| hlambdaLC | 5′-CACACYAGTGTGGCCTTGTTGGCTTG-3′ | 8 |
| rev | | |

For sequences 9 to 69, see the examples section above. For sequences 70 to 78 see FIG. 5.

### REFERENCES

“Adaptive antibody diversification through N-linked glycosylation of the immunoglobulin variable region.” van de Bovenkamp F S, Derksen N I L, Ooijevaar-de Heer P, van Schie K A, Kruithof S, Berkowska M A, van der Schoot C E, Uspeert H, van der Burg M, Gils A, Hafkenscheid L, Toes R E M, Rombouts Y, Plomp R, Wuhrer M, van Ham S M, Vidarsson G, Rispens T. Proc Natl Acad Sci USA. 2018 Feb. 20; 115(8):1901-1906.
“The role of MuSK in synapse formation and neuromuscular disease.” Burden S J, Yumoto N, Zhang W. Cold Spring Harb Perspect Biol. 2013 May 1; 5(5):a009167
“Fundamental Molecules and Mechanisms for Forming and Maintaining Neuromuscular Synapses.” Burden S J, Huijbers M G, Remedio L. Int J Mol Sci. 2018 Feb. 6; 19(2).
“Clinical correlates with anti-MuSK antibodies in generalized seronegative myasthenia gravis.” Evoli A, Tonali P A, Padua L, Monaco M L, Scuderi F, Batocchi A P, Marino M, Bartoccioni E. Brain. 2003 October; 126(Pt 10):2304-11.
“Synapse disassembly and formation of new synapses in postnatal muscle upon conditional inactivation of MuSK.” Hesser B A, Henschel O, Witzemann V. Mol Cell Neurosci. 2006 March; 31(3):470-80.
“Auto-antibodies to the receptor tyrosine kinase MuSK in patients with myasthenia gravis without acetylcholine receptor antibodies.” Hoch W, McConville J, Helms S, Newsom-Davis J, Melms A, Vincent A. Nat Med. 2001 March; 7(3):365-8.
“Longitudinal epitope mapping in MuSK myasthenia gravis: implications for disease severity.” Huijbers M G, Vink A F, Niks E H, Westhuis R H, van Zwet E W, de Meel R H, Rojas-Garcia R, Diaz-Manera J, Kuks J B, Klooster R, Straasheijm K, Evoli A, IIla I, van der Maarel S M, Verschuuren J J. J Neuroimmunol. 2016 Feb. 15; 291:82-8
“MuSK IgG4 autoantibodies cause myasthenia gravis by inhibiting binding between MuSK and Lrp4.” Huijbers M G, Zhang W, Klooster R, Niks E H, Friese M B, Straasheijm K R, Thijssen P E, Vrolijk H, Plomp J J, Vogels P, Losen M, Van der Maarel S M, Burden S J, Verschuuren J J. Proc Natl Acad Sci USA. 2013 Dec. 17; 110(51):20783-8.
“Muscle-specific kinase myasthenia gravis IgG4 autoantibodies cause severe neuromuscular junction dysfunction in mice.” Klooster R, Plomp J J, Huijbers M G, Niks E H, Straasheijm K R, Detmers F J, Hermans P W, Sleijpen K, Verrips A, Losen M, Martinez-Martinez P, De Baets M H, van der Maarel S M, Verschuuren J J. Brain. 2012 April; 135(Pt 4):1081-101
“MuSK myasthenia gravis IgG4 disrupts the interaction of LRP4 with MuSK but both IgG4 and IgG1-3 can disperse preformed agrin-independent AChR clusters.” Koneczny I, Cossins J, Waters P, Beeson D, Vincent A. PLoS One. 2013 Nov. 7; 8(11):e80695.
“IgG4 autoantibodies against muscle-specific kinase undergo Fab-arm exchange in myasthenia gravis patients.” Koneczny I, Stevens J A, De Rosa A, Huda S, Huijbers M G, Saxena A, Maestri M, Lazaridis K, Zisimopoulou P, Tzartos S, Verschuuren J, van der Maarel S M, van Damme P, De Baets M H, Molenaar P C, Vincent A, Ricciardi R, Martinez-Martinez P, Losen M. J Autoimmun. 2017 February; 77:104-115.
“ARTISAN PCR: rapid identification of full-length immunoglobulin rearrangements without primer binding bias.” Koning M T, Kielbasa S M, Boersma V, Buermans H P J, van der Zeeuw S A J, van Bergen C A M, Cleven A H G, Kluin P M, Griffioen M, Navarrete M A, Veelken H. Br J Haematol. 2017 September; 178(6):983-986.
“The Immunobiology of Immunoglobulin G4.” Lighaam L C, Rispens T. Semin Liver Dis. 2016 August; 36(3):200-15.
“Phenotypic differences between IgG4+ and IgG1+B cells point to distinct regulation of the IgG4 response.” Lighaam L C, Vermeulen E, Bleker Td, Meijlink K J, Aalberse R C, Barnes E, Culver E L, van Ham S M, Rispens T. J Allergy Clin Immunol. 2014 January; 133(1):267-70.e1-6.
“Detection and characterization of MuSK antibodies in seronegative myasthenia gravis.” McConville J, Farrugia M E, Beeson D, Kishore U, Metcalfe R, Newsom-Davis J, Vincent A. Ann Neurol. 2004 April; 55(4):580-4.
“Anti-inflammatory activity of human IgG4 antibodies by dynamic Fab arm exchange.” van der Neut Kolfschoten M, Schuurman J, Losen M, Bleeker W K, Martinez-Martinez P, Vermeulen E, den Bleker T H, Wiegman L, Vink T, Aarden L A, De Baets M H, van de Winkel J G, Aalberse R C, Parren P W. Science. 2007 Sep. 14; 317(5844):1554-7.
“Collagen Q and anti-MuSK autoantibody competitively suppress agrin/LRP4/MuSK signaling.” Otsuka K, Ito M, Ohkawara B, Masuda A, Kawakami Y, Sahashi K, Nishida H, Mabuchi N, Takano A, Engel A G, Ohno K. Sci Rep. 2015 Sep. 10; 5:13928.
“Crystal structure of the agrin-responsive immunoglobulin-like domains 1 and 2 of the receptor tyrosine kinase MuSK.” Stiegler A L, Burden S J, Hubbard S R. J Mol Biol. 2006 Dec. 1; 364(3):424-33.
“Agrin binds to the N-terminal region of Lrp4 protein and stimulates association between Lrp4 and the first immunoglobulin-like domain in muscle-specific kinase (MuSK).” Zhang W, Coldefy A S, Hubbard S R, Burden S J. J Biol Chem. 2011 Nov. 25; 286(47):40624-30.

1. A binding agent comprising one or more binding regions, wherein only one binding region specifically binds to an Ig-like 1 domain of a MuSK protein, for use in treating a condition, disorder and/or symptom which is alleviated by the inhibition of neuromuscular transmission in a subject.

2. The binding agent for use according to claim 1, wherein the binding agent is a binding protein.

3. The binding agent for use according to claim 2, wherein the region that specifically binds to an Ig-like 1 domain of the MuSK protein is a variable region.

4. The binding agent for use according to claim 1, wherein the MuSK protein is a human MuSK protein.

5. The binding agent for use according to claim 1, wherein the binding agent is monovalent.

6. The binding agent for use according to claim 1, wherein the binding agent is bivalent or trivalent.

7. The binding agent for use according to claim 1, wherein the binding agent is an antibody.

8. The binding agent for use according to claim 7, wherein the antibody is a monoclonal antibody.

9. The binding agent for use according to claim 7, wherein the antibody is a human antibody or a humanised antibody.

10. The binding agent for use according to claim 7, wherein the antibody is selected from a Fab, bi-specific Fab₂, tri-specific Fab₃, scFv, bi-specific di-scFv, bi-specific scFv-Fc, bi-specific diabody, a tri-specific triabody, a single domain antibody or a bi-specific minibody.

11. The binding agent for use according to claim 7, wherein the antibody is an IgG.

12. (canceled)

13. The binding agent for use according to claim 11, wherein the IgG is an lgG4 variant with a reduced ability for or an inability for Fab-arm exchange in vivo.

14. (canceled)

15. The binding agent for use according to claim 13, wherein the lgG4 variant comprises an lgG4 constant region comprising an amino acid substitution at amino acid position 228 and/or an amino acid substitution at amino acid position 409 and/or an amino acid substitution at amino acid position 405 of the heavy chain numbered according to the EU index.

16. The binding agent for use according to claim 1, wherein the subject is a human.

17. The binding agent for use according to claim 1, wherein the condition, disorder and/or symptom is selected from:
(i) an external appearance distorted due to excessive muscular activity; or
(ii) a condition, disorder or symptom resulting from excessive muscular activity, including dystonias, facial spasms, strabismus, cerebral palsy, stuttering, chronic tension headaches, spasms of the inferior constrictor of the pharynx, pain, migraine, involuntary spasms, muscle spasticity, strabismus, occupational cramps, anal fissures, brusism, and any combination thereof.

18. The binding agent for use according to claim 1, wherein the binding agent is an antibody or an antibody comprising one or more binding regions, wherein the only one binding region that specifically binds to an Ig-like 1 domain of a MuSK protein has a sequence selected from:
a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO: 13;
b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;
c) a VH CDR1 comprising SEQ ID NO:26, a VH CDR2 comprising SEQ ID NO:27, and a VH CDR3 comprising SEQ ID NO:28, optionally wherein the binding region comprises a heavy chain variable domain comprising SEQ ID NO: 25;
d) a VH CDR1 comprising SEQ ID NO:30, a VH CDR2 comprising SEQ ID NO:31, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;
e) a VH CDR1 comprising SEQ ID NO:38, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;
f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;
g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or
h) combinations of any of the above.

19. A method of preventing, regulating or reducing skin wrinkling in a subject, the method comprising administering a binding agent comprising one or more binding regions, wherein only one binding region specifically binds to an Ig-like 1 domain of a MuSK protein, to the subject.

20. A method of treating or preventing a condition, disorder and/or symptom which is alleviated by the inhibition of neuromuscular transmission; the method comprising administering a binding agent comprising one or more binding regions, wherein only one binding region specifically binds to an Ig-like 1 domain of a MuSK protein, to a subject.

21-36. (canceled)

37. The method according to claim 19, wherein the binding agent is an antibody and the only one binding region that specifically binds to an Ig-like 1 domain of a MuSK protein has a sequence selected from:
a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO: 13;
b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;
c) a VH CDR1 comprising SEQ ID NO:26, a VH CDR2 comprising SEQ ID NO:27, and a VH CDR3 comprising SEQ ID NO:28, optionally wherein the binding region comprises a heavy chain variable domain comprising SEQ ID NO: 25;
d) a VH CDR1 comprising SEQ ID NO:30, a VH CDR2 comprising SEQ ID NO:31, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;
e) a VH CDR1 comprising SEQ ID NO:38, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;
f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;
g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or
h) combinations of any of the above.

38. The method according to claim 20, wherein the binding agent is an antibody and the only one binding region that specifically binds to an Ig-like 1 domain of a MuSK protein has a sequence selected from:
a) a VH CDR1 comprising SEQ ID NO:10, a VH CDR2 comprising SEQ ID NO:11, a VH CDR3 comprising SEQ ID NO:12, a VL CDR1 comprising SEQ ID NO:14, a VL CDR2 comprising SEQ ID NO:15, and a VL CDR3 comprising SEQ ID NO: 16; optionally wherein the binding region comprises a heavy chain variable domain comprising SEQ ID NO: 9 and a light chain variable domain comprising SEQ ID NO: 13;
b) a VH CDR1 comprising SEQ ID NO:18, a VH CDR2 comprising SEQ ID NO:19, a VH CDR3 comprising SEQ ID NO:20, a VL CDR1 comprising SEQ ID NO:22, a VL CDR2 comprising SEQ ID NO:23, and a VL CDR3 comprising SEQ ID NO: 24, optionally wherein the binding region comprises a heavy chain variable domain comprising SEQ ID NO: 17 and a light chain variable domain comprising SEQ ID NO:21;
c) a VH CDR1 comprising SEQ ID NO:26, a VH CDR2 comprising SEQ ID NO:27, and a VH CDR3 comprising SEQ ID NO\hat{ }S, optionally wherein the binding region comprises a heavy chain variable domain comprising SEQ ID NO: 25;
d) a VH CDR1 comprising SEQ ID NO:30, a VH CDR2 comprising SEQ ID NO\hat{ }I, a VH CDR3 comprising SEQ ID NO:32, a VL CDR1 comprising SEQ ID NO:34, a VL CDR2 comprising SEQ ID NO:35, and a VL CDR3 comprising SEQ ID NO: 36, optionally wherein the binding region comprises a heavy chain variable domain comprising SEQ ID NO: 29 and a light chain variable domain comprising SEQ ID NO: 33;
e) a VH CDR1 comprising SEQ ID NO\hat{ }S, a VH CDR2 comprising SEQ ID NO:39, a VH CDR3 comprising SEQ ID NO:40, a VL CDR1 comprising SEQ ID NO:42, a VL CDR2 comprising SEQ ID NO:43, and a VL CDR3 comprising SEQ ID NO: 44, optionally wherein the binding region comprises a heavy chain variable domain comprising SEQ ID NO: 37 and a light chain variable domain comprising SEQ ID NO:41;
f) a VH CDR1 comprising SEQ ID NO:46, a VH CDR2 comprising SEQ ID NO:47, a VH CDR3 comprising SEQ ID NO:48, a VL CDR1 comprising SEQ ID NO:50, a VL CDR2 comprising SEQ ID NO:51, and a VL CDR3 comprising SEQ ID NO: 52, optionally wherein the binding region comprises a heavy chain variable domain comprising SEQ ID NO: 45 and a light chain variable domain comprising SEQ ID NO: 49;
g) a VH CDR1 comprising SEQ ID NO:54, a VH CDR2 comprising SEQ ID NO:55, a VH CDR3 comprising SEQ ID NO:56, a VL CDR1 comprising SEQ ID NO:58, a VL CDR2 comprising SEQ ID NO:59, and a VL CDR3 comprising SEQ ID NO: 60, optionally wherein the binding region comprises a heavy chain variable domain comprising SEQ ID NO: 53 and a light chain variable domain comprising SEQ ID NO: 57; or
h) combinations of any of the above.


SEQUENCE LISTING (78 sequences)

SEQ ID NO: 1 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 2 - protein, 90 aa, Homo sapiens
PVITTPLETVDALVEEVATFMCAVESYPQPEISWTRNKILIKLFDTRYSIRENGQLLTILSVEDSDDGIYCCTANNGVGGAVESCGALQV

SEQ ID NO: 3 - DNA, 67 bp, Artificial Sequence
  Primer Oligo-dT40VN
  misc_feature (67)..(67): n is a, c, g, or t
aagcagtggtatcaacgcagagtacttttttttttttttttttttttttttttttttttttttttvn

SEQ ID NO: 4 - DNA, 30 bp, Artificial Sequence
  Primer BC-TSO
  misc_feature (30)..(31): riboguanosines
  misc_feature (32)..(32): LNA-modified guanosine
aagcagtggtatcaacgcagagtacatggg

SEQ ID NO: 5 - DNA, 31 bp, Artificial Sequence
  General Fw
cttaagcagtggtatcaacgcagagtacatg

SEQ ID NO: 6 - DNA, 23 bp, Artificial Sequence
  IgG HC rev
ggaaggtgtgcacgccgctggtc

SEQ ID NO: 7 - DNA, 19 bp, Artificial Sequence
  Primer hkappaLC rev
ctgatgggtgacttcgcmg

SEQ ID NO: 8 - DNA, 26 bp, Artificial Sequence
  Primer hlambdaLC rev
cacacyagtgtggccttgttggcttg

SEQ ID NO: 9 - protein, 121 aa, Homo sapiens
EVQLVESGGGLVKPGESLRLSCAASGFNFSTYTMHWVRQAPGKGLEWVSSISSRSAYKYYADSVKGRFTISRDIAKNSLYLQMNSLRAEDTAVYYCARDFFQLGPPRFDSWGQGTLVTVSS

SEQ ID NO: 10 - protein, 8 aa, Homo sapiens
GFNFSTYT

SEQ ID NO: 11 - protein, 8 aa, Homo sapiens
ISSRSAYK

SEQ ID NO: 12 - protein, 14 aa, Homo sapiens
ARDFFQLGPPRFDS

SEQ ID NO: 13 - protein, 107 aa, Homo sapiens
DIQMTQSPSSLSASIGDRVTISCRASQRISSFLNWYQQKPGKPPNLLIYGASNLQSGVPSRFSGSGSGTDFTLTITSLQPEDYAIYYCQQSYSPMYTFGQGTSLEIK

SEQ ID NO: 14 - protein, 6 aa, Homo sapiens
QRISSF

SEQ ID NO: 15 - protein, 3 aa, Homo sapiens
GAS

SEQ ID NO: 16 - protein, 9 aa, Homo sapiens
QQSYSPMYT

SEQ ID NO: 17 - protein, 121 aa, Homo sapiens
EVQLVESGGGLVKPGGSLRLSCAASGFTFSSYTMDWVRQAPGKGLEWVSSIGSNGDYIYYADSVRGRFTISRDNAKNSLYLQMNSLRPDDTADYYCARGQLAVAGTHFDYWGRGSLVTVSS

SEQ ID NO: 18 - protein, 8 aa, Homo sapiens
GFTFSSYT

SEQ ID NO: 19 - protein, 8 aa, Homo sapiens
IGSNGDYI

SEQ ID NO: 20 - protein, 14 aa, Homo sapiens
ARGQLAVAGTHFDY

SEQ ID NO: 21 - protein, 107 aa, Homo sapiens
DIQMTQSPSSLSASVGDRVTISCRASQKVNKYVNWYQQTPGKAPRLLIYAASTLQSGVPSRFSGSGSGANFTLTISGLQPEDFAIYFCQQSYSPLCTFGQGTKLEIK

SEQ ID NO: 22 - protein, 6 aa, Homo sapiens
QKVNKY

SEQ ID NO: 23 - protein, 3 aa, Homo sapiens
AAS

SEQ ID NO: 24 - protein, 9 aa, Homo sapiens
QQSYSPLCT

SEQ ID NO: 25 - protein, 121 aa, Homo sapiens
EVQLVESGGGLVKPGGSLRLSCAASGFTFSDFTMNWVRQAPGQGLEWVSSIGSSGTFIYYAASVRGRFTISRDNAQDLLSLQMNSLRADDTATYFCARGRIAVAGTHFDLWGQGTLVTVSS

SEQ ID NO: 26 - protein, 8 aa, Homo sapiens
GFTFSDFT

SEQ ID NO: 27 - protein, 8 aa, Homo sapiens
IGSSGTFI

SEQ ID NO: 28 - protein, 14 aa, Homo sapiens
ARGRIAVAGTHFDL

SEQ ID NO: 29 - protein, 121 aa, Homo sapiens
QVQLVQSGAVVAKPGASVQVSCKTSGYTFTGQYLHWVRQAPGQGLEWIGWINPSSGVTKFAEKFEGRATMTRDTSITTAYIDLRSLRSDDTATYYCATLSLGVYYVGMVAWGQGTLVTVSS

SEQ ID NO: 30 - protein, 8 aa, Homo sapiens
GYTFTGQY

SEQ ID NO: 31 - protein, 8 aa, Homo sapiens
INPSSGVT

SEQ ID NO: 32 - protein, 14 aa, Homo sapiens
ATLSLGVYYVGMVA

SEQ ID NO: 33 - protein, 108 aa, Homo sapiens
SYELTQPPSVSVSPGQTATITCSGDGLAQQHVYWFQQRPGQAPLLIIYKDIERPSGIPERFSGSSSGTTAMLTISGVQAEDEADYYCQSGDRTATSVLFGGGTKMTVL

SEQ ID NO: 34 - protein, 6 aa, Homo sapiens
GLAQQH

SEQ ID NO: 35 - protein, 3 aa, Homo sapiens
KDI

SEQ ID NO: 36 - protein, 11 aa, Homo sapiens
QSGDRTATSVL

SEQ ID NO: 37 - protein, 121 aa, Homo sapiens
EDLLVESGGGLVKPGQSLTLSCAASGFDFSASTMAWVRQAPGKGLEWVASVSGDSHHIYYADSLKGRFTLSRDNARNSFFLEMNSLRAEDTAVYYCARERLLRLGVGFDSWGQGSLVAVSS

SEQ ID NO: 38 - protein, 8 aa, Homo sapiens
GFDFSAST

SEQ ID NO: 39 - protein, 8 aa, Homo sapiens
VSGDSHHI

SEQ ID NO: 40 - protein, 14 aa, Homo sapiens
ARERLLRLGVGFDS

SEQ ID NO: 41 - protein, 107 aa, Homo sapiens
DIHMTQSPSSLSSSVGDRVTMTCRASQRISGFVNWYQQKPGRAPTLLISAASTLQSGVPSRFSGSASGTDFTLTISGLQPEDSAIYYCQQSYSPLYTFGQGTKVEIK

SEQ ID NO: 42 - protein, 6 aa, Homo sapiens
QRISGF

SEQ ID NO: 43 - protein, 3 aa, Homo sapiens
AAS

SEQ ID NO: 44 - protein, 9 aa, Homo sapiens
QQSYSPLYT

SEQ ID NO: 45 - protein, 121 aa, Homo sapiens
EVQLMESGGGLVKPGGSLRLSCTASGFTFSSYTMTWVRQAPGKGLEWVSSISSGGHYIYYTDSLKGRFIISRDNAKNSVFLQMNNLRAEDTATYYCARERLLRLGVGFDFWGQGSLVTVSS

SEQ ID NO: 46 - protein, 8 aa, Homo sapiens
GFTFSSYT

SEQ ID NO: 47 - protein, 8 aa, Homo sapiens
ISSGGHYI

SEQ ID NO: 48 - protein, 14 aa, Homo sapiens
ARERLLRLGVGFDF

SEQ ID NO: 49 - protein, 107 aa, Homo sapiens
DIQMTQSPSSLSASEGDRVTMTCRASQSISGYLNWYQQKPGKAPKLLIYAASTLQSGVPSRFSGSSSGTEFTLSISSLQPEDFATYYCQQSYSALYTFGQGTRVEIK

SEQ ID NO: 50 - protein, 6 aa, Homo sapiens
QSISGY

SEQ ID NO: 51 - protein, 3 aa, Homo sapiens
AAS

SEQ ID NO: 52 - protein, 9 aa, Homo sapiens
QQSYSALYT

SEQ ID NO: 53 - protein, 117 aa, Homo sapiens
EVQLVESGGGLVQPGGSLRLSCAASGFTFSSYWMHWVRQVPGKGLVWVSRLNEDGSTTNYADSVKGRFTISRDNAKYTLYLQMNSLRFEDTAVYYCVSDLSGKDEHWGQGTLVTVSS

SEQ ID NO: 54 - protein, 8 aa, Homo sapiens
GFTFSSYW

SEQ ID NO: 55 - protein, 8 aa, Homo sapiens
LNEDGSTT

SEQ ID NO: 56 - protein, 10 aa, Homo sapiens
VSDLSGKDEH

SEQ ID NO: 57 - protein, 112 aa, Homo sapiens
DIVMTQSPLSLPVTPGEPASISCRSTQSLLHSNGYYWLDWYLQKPGQSPQLLVYLGFNRASGVPDRFSGSGSGTDFTLKISRVEAEDVGVYYCMQGLQTPYTFGQGTTLEIK

SEQ ID NO: 58 - protein, 11 aa, Homo sapiens
QSLLHSNGYYW

SEQ ID NO: 59 - protein, 3 aa, Homo sapiens
LGF

SEQ ID NO: 60 - protein, 9 aa, Homo sapiens
MQGLQTPYT

SEQ ID NO: 61 - protein, 399 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 62 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 63 - protein, 377 aa, Homo sapiens
  misc_feature (1)..(1): Xaa can be any naturally occurring amino acid
XSTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 64 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 204 aa, Artificial Sequence
  11-3D9 clone amino acid sequence (translated from cDNA)
GDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVTR

SEQ ID NO: 66 - protein, 202 aa, Artificial Sequence
  11-8G4 clone amino acid sequence (translated from cDNA)
GDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTV

SEQ ID NO: 67 - protein, 201 aa, Artificial Sequence
  13-3D10 clone amino acid sequence (translated from cDNA)
KRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVT

SEQ ID NO: 68 - protein, 246 aa, Artificial Sequence
  13-4D3 clone amino acid sequence (translated from cDNA)
RVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTV

SEQ ID NO: 69 - protein, 200 aa, Artificial Sequence
  13-3B5 clone amino acid sequence (translated from cDNA)
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVTR

SEQ ID NO: 70 - protein, 203 aa, Artificial Sequence
  Synthetic polypeptide
  MISC_FEATURE (1)..(203): 11-3D9
GDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVT

SEQ ID NO: 71 - protein, 201 aa, Artificial Sequence
  Synthetic polypeptide
  MISC_FEATURE (1)..(201): 11-8G4
GDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLT

SEQ ID NO: 72 - protein, 199 aa, Artificial Sequence
  Synthetic polypeptide
  MISC_FEATURE (1)..(199): 13-3B5
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVT

SEQ ID NO: 73 - protein, 200 aa, Artificial Sequence
  Synthetic polypeptide
  MISC_FEATURE (1)..(200): 13-3D10
KRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTV

SEQ ID NO: 74 - protein, 245 aa, Artificial Sequence
  Synthetic polypeptide
  MISC_FEATURE (1)..(245): 13-4D3
RVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLT

SEQ ID NO: 75 - protein, 334 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEE

SEQ ID NO: 76 - protein, 324 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSP

SEQ ID NO: 77 - protein, 375 aa, Homo sapiens
  misc_feature (1)..(1): Xaa can be any naturally occurring amino acid
XSTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSP

SEQ ID NO: 78 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL